SEQUENCE LISTING (32 sequences)

SEQ ID NO: 1 - protein, 318 aa, Arabidopsis thaliana
MKCNDKMVVIMGATGSGKSSLSVDLALHFKAEIINSDKMQFYDGLKITTNQSTIEDRRGVPHHLLGELNPEAGEVTAAEFRVMAAEAISEITQRKKLPILAGGSNSYIHALLAKSYDPENYPFSDHKGSICSELKYDCCFIWIDVDQSVLFEYLSLRLDLMMKSGMFEEIAEFHRSKKAPKEPLGIWKAIGVQEFDDYLKMYKWDNDMDKWDPMRKEAYEKAVRAIKENTFQLTKDQITKINKLRNAGWDIKKVDATASFREAIRAAKEGEGVAEMQRKIWNKEVLEPCVKIVRSHLDQPINYYYYYFYLLKRFLSLN

SEQ ID NO: 2 - protein, 518 aa, Arabidopsis thaliana
MLLTILKSLLVIFVTTILRVLYDTISCYWLTPRRIKKIMEQQGVTGPKPRPLTGNILEISAMVSQSASKDCDSIHHDIVGRLLPHYVAWSKQYGKRFIVWNGTDPRLCLTETELIKELLMKHNGVSGRSWLQQQGTKNFIGRGLLMANGQDWHHQRHLAAPAFTGERLKGYARHMVECTSKLVERLRKEVGEGANEVEIGEEMHKLTADIISRTKFGSSFEKGKELFNHLTVLQRRCAQATRHLCFPGSRFLPSKYNREIKSLKKEVERLLIEIIQSRRDCAEMGRSSTHGDDLLGLLLNEMDIDKNNNNNNNNLQLIMDECKTFFFAGHETTALLLTWTTMLLADNPTWQEKVREEVREVFGRNGLPSVDQLSKLTSLSKVINESLRLYPPATLLPRMAFEDLKLGDLTIPKGLSIWIPVLAIHHSEELWGKDANQFNPERFGGRPFASGRHFIPFAAGPRNCIGQQFALMEAKIILATLISKFNFTISKNYRHAPIVVLTIKPKYGVQVILKPLVS

SEQ ID NO: 3 - protein, 692 aa, Arabidopsis thaliana [sequence omitted]

SEQ ID NO: 4 - protein, 217 aa, Arabidopsis thaliana
MEETKSRFKRICVFCGSSSGKKPSYQEAAIQLGNELVERRIDLVYGGGSVGLMGLVSQAVHHGGRHVLGVIPKTLMPREITGETIGEVKAVADMHQRKAEMARQADAFIALPGGYGTLEELLEVITWAQLGIHRKPVGLLNVDGYYNSLLTFIDKAVDEGFISPMARRIIVSAPNAKELVRQLEEYEPEFDEITSKLVWDEVDRISYVPGSEVATAT

SEQ ID NO: 5 - protein, 527 aa, Saccharomyces cerevisiae
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 6 - protein, 623 aa, Xanthophyllomyces dendrorhous
MGHKNQNVNLRLSLERDTGLLRYQREQAYQAQSQILAPISPVSVAPVVSNGNGNASKSIEKPMPRLVVPNGPRSLPESPPSTTESTPVNKVIIGGPSDRPALDGLANGNGAVPLDKQTVLGMRSIEECEEIMKSGLGPYSLNDEELILLTQKGKIPPYSLEKALQNCERAVKIRRAVISRASVTKTLETSDLPMKDYDYSKVMGACCENVVGYMPLPVGIAGPLNIDGEVVPIPMATTEGTLVASTSRGCKALNAGGGVTTVITQDAMTRGPVVDFPSVSQAAQAKRWLDSVEGMEVMAASFNSTSRFARLQSIKCGMAGRSLYIRLATSTGDAMGMNMAGKGTEKALETLSEYFPSMQILALSGNYCIDKKPSAINWIEGRGKSVVAESVIPGAIVKSVLKTTVADLVNLNIKKNLIGSAMAGSIGGFNAHASNILTSIFLATGQDPAQNVESSMCMTLMEAVNDGKDLLITCSMPAIECGTVGGGTFLPPQNACLQMLGVAGAHPDSPGHNARRLARIIAASVMAGELSLMSALAAGHLIKAHMKHNRSTPSTPLPVSPLATRPNTPSHRSIGLLTPMTSSASVASMFSGFGSPSTSSLKTVGSMACVRERGDETSVNVDA

SEQ ID NO: 7 - protein, 288 aa, Saccharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 8 - DNA, 957 bp, synthetic construct
  source 1..957: mol_type = other DNA
atgaagtgtaacgataagatggttgttattatgggtgctactggttctggtaaatcttcattgtcagttgatttggctttacatttcaaagcagaaattattaactctgataagatgcaattctacgatggtttgaagatcactacaaaccaatcaacaattgaagatagaagaggtgttccacatcatttgttgggtgaattaaatccagaagctggtgaagttactgctgcagaattcagagttatggctgcagaagctatctctgaaatcacacaaagaaagaaattgccaatcttggcaggtggttctaactcatacatccatgctttgttagcaaaatcttacgatccagaaaactacccattttcagatcataagggttctatctgttcagaattgaagtacgattgttgtttcatctggatcgatgttgatcaatctgttttatttgaatatttgtcattgagattggatttgatgatgaaatctggcatgttcgaagaaatcgctgaattccatagatctaagaaagcaccaaaagaaccattgggtatttggaaagctattggtgttcaagaattcgatgattacttaaagatgtacaagtgggataatgatatggataaatgggatccaatgagaaaagaagcatacgaaaaagctgttagagcaattaaagaaaatacttttcaattgacaaaggatcaaatcactaaaattaataagttaagaaatgctggttgggatattaagaaagttgatgctacagcatcttttagagaagctattagagctgcaaaagaaggtgaaggtgttgcagaaatgcaaagaaagatctggaataaggaagttttggaaccatgtgttaagatcgttagatctcatttggatcaaccaattaattattactattactatttttatttgttaaaaagatttttgtcattaaattaa

SEQ ID NO: 9 - DNA, 1556 bp, synthetic construct
  source 1..1556: mol_type = other DNA
atgttgttgactatcttaaaatcattgttagttatttttgttactacaattttgagagttttatatgatactatttcttgttactggttgacaccaagaagaattaagaaaattatggaacaacaaggtgttactggtccaaagccaagaccattaactggtaacatcttggaaatctcagctatggtttctcaatcagcatctaaggattgtgattctatccatcatgatatcgttggtagattgttaccacattatgttgcttggtctaagcaatacggtaaaagattcattgtttggaatggtactgatccaagattgtgtttaactgaaacagaattgattaaagaattgttgatgaagcataatggtgtttcaggtagatcttggttgcagcaacaaggtactaaaaatttcatcggtagaggtttgttgatggcaaatggtcaagattggcatcatcaaagacatttggctgcaccagcttttactggtgaaagattgaaaggttatgcaagacatatggttgaatgtacatcaaagttggttgaaagattgagaaaagaagttggtgaaggtgctaatgaagttgaaatcggtgaagaaatgcataagttgactgcagatatcatctctagaacaaaatttggttcttcattcgaaaagggtaaagaattgtttaatcatttgactgttttacaaagaagatgtgctcaagcaacaagacatttgtgttttccaggttcaagatttttgccatctaagtacaacagagaaattaaatcattgaagaaagaagttgaaagattgttaattgaaattattcaatctagaagagattgtgctgaaatgggtagatcttcaactcatggtgacgatttgttaggtttgttattgaatgaaatggatattgataagaacaataataacaacaacaacaatttgcaattaattatggatgaatgtaaaacatttttctttgctggtcatgaaactacagcattattgttgacttggactacaatgttgttagctgataatccaacatggcaagaaaaagttagagaagaagttagagaagtttttggtagaaatggtttgccatcagttgatcaattgtctaagttgacttcattgtctaaagttattaatgaatcattgagattatatccaccagctactttgttaccaagaatggcatttgaagatttgaaattgggtgacttgacaattccaaaaggtttgtcaatttggattccagttttggctatccatcattctgaagaattgtggggtaaagatgcaaatcaattcaatccagaaagatttggtggtagaccatttgcttctggtagacattttattccatttgctgcaggtccaagaaactgtatcggtcaacaattcgctttgatggaagcaaaaattattttagctactttgatttcaaagtttaatttcacaatctctaaaaattacagacatgcaccaatcgttgttttgacaattaaaccaaagtacggtgttcaagttattttaaaaccattggtttctta

SEQ ID NO: 10 - DNA, 2079 bp, synthetic construct
  source 1..2079: mol_type = other DNA
atgacttctgctttgtatgcatcagatttgtttaaacaattaaaatctatcatgggtacagattctttgtcagatgatgttgttttagttattgctactacatcattggctttagttgcaggtttcgttgttttgttgtggaagaaaactacagctgatagatctggtgaattgaagccattgatgatcccaaaatcattgatggctaaggatgaagatgatgatttggatttgggttctggtaaaactagagtttcaattttctttggtactcaaacaggtactgctgaaggtttcgctaaggcattgtctgaagaaattaaagcaagatacgaaaaggctgcagttaaagttattgatttggatgattatgctgcagatgatgatcaatacgaagaaaagttgaagaaagaaactttggctttcttttgtgttgcaacttatggtgacggtgaaccaacagataacgctgcaagattctacaagtggttcacagaagaaaacgaaagagatatcaagttgcaacaattagcttatggtgtttttgcattgggtaacagacaatacgaacatttcaataagatcggtatcgttttggatgaagaattatgcaagaaaggtgctaagagattgattgaagttggtttaggtgacgatgatcaatctatcgaagatgatttcaacgcttggaaggaatctttgtggtcagaattggataagttgttgaaggatgaagatgataagtctgttgctacaccatatactgcagttattccagaatacagagttgttactcatgatccaagattcactacacaaaaatctatggaatcaaacgttgctaacggtaacactacaatcgatatccatcatccatgtagagttgatgttgctgttcaaaaggaattgcatacacatgaatctgatagatcatgtatccatttggaattcgatatctctagaacaggtatcacttatgaaacaggtgaccatgttggtgtttacgctgaaaatcatgttgaaattgttgaagaagctggtaaattgttaggtcattctttggatttggttttctctattcatgctgataaagaagatggttctccattagaatcagcagttccaccaccatttccaggtccatgtactttgggtacaggtttagctagatatgcagatttgttaaatccaccaagaaaatctgctttggttgcattagctgcatacgctactgaaccatcagaagcagaaaaattgaaacatttgacatctccagatggtaaagatgaatattcacaatggattgttgcttctcaaagatcattgttagaagttatggctgcatttccatctgctaaaccaccattgggtgttttctttgctgcaattgcaccaagattgcaaccaagatactactcaatctcttcatctccaagattggctccatctagagttcatgttacttcagcattagtttacggtccaacaccaactggtagaattcataaaggtgtttgttctacatggatgaaaaatgctgttccagcagaaaaatctcatgaatgttcaggtgctccaattttcattagagcatcaaacttcaagttgccatctaacccatcaactccaattgttatggttggtccaggtacaggtttagctccttttagaggtttcttgcaagaaagaatggcattaaaagaagatggtgaagaattgggttcatctttgttgtttttcggttgtagaaatagacaaatggatttcatctatgaagatgaattgaacaactttgttgatcaaggtgttatttctgaattgatcatggctttttcaagagaaggtgcacaaaaggaatacgttcaacataagatgatggaaaaagctgcacaagtttgggatttgattaaagaagaaggttatttgtacgtttgtggtgacgctaagggtatggcaagagatgttcatagaactttgcatacaatcgttcaagaacaagaaggtgtttcatcttcagaagctgaagcaattgttaagaaattgcaaactgaaggtagatatttgagagatgtttggtaa

SEQ ID NO: 11 - DNA, 654 bp, synthetic construct
  source 1..654: mol_type = other DNA
atggaagaaactaaatcaagattcaaaagaatctgtgttttctgtggttcttcatctggtaaaaagccatcttatcaagaagctgcaatccaattgggtaacgaattggttgaaagaagaatcgatttggtttacggtggtggttcagttggtttgatgggtttagtttctcaagctgttcatcatggtggtagacatgttttgggtgttattccaaagacattgatgccaagagaaattactggtgaaacaattggtgaagttaaagctgttgcagatatgcatcaaagaaaagctgaaatggcaagacaagctgatgcttttattgctttgccaggtggttatggtactttggaagaattgttggaagttattacatgggcacaattgggtatccatagaaagccagttggtttgttgaacgttgatggttactacaactcattgttgacttttattgataaggctgttgatgaaggttttatttcaccaatggcaagaagaatcattgtttctgctccaaacgcaaaggaattggttagacaattggaagaatacgaaccagaattcgatgaaatcacatctaagttggtttgggatgaagttgatagaatttcatacgttccaggttctgaagttgctactgcaacataa

SEQ ID NO: 12 - DNA, 1584 bp, Saccharomyces cerevisiae
  source 1..1584: mol_type = other DNA
atggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 13 - DNA, 1872 bp, Xanthophyllomyces dendrorhous
  source 1..1872: mol_type = other DNA
atgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctga

SEQ ID NO: 14 - DNA, 867 bp, Saccharomyces cerevisiae
  source 1..867: mol_type = other DNA
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataa

SEQ ID NO: 15 - DNA, 1917 bp, synthetic construct
  source 1..1917: mol_type = other DNA
ccacagagaacaggagattacaagatccagatcaccaaaatgccggaacacaggctgaaaaactccaagcagccgcacacccgatttttagccagcagattcatacctaactccaatgaattgcagcatatatcctgcaataacacattaaggatcgggccttggacggccgaatatatgattatagtccttttgtcgccaaaaagtcgcctcgaggtcacgtgcatccgaaatcaccccctgagcacttcccgcccagaaaacagggaaggggcggcaccagcaggcaccggcattcactcgtccgtgcggcgtggtctgtgccgttcaggggcctgcgtgcctgtgatgatatcgctgcagggcaccgagtcggagaggaccactccgagccgaggttagttaagccattctctacgctaaaaaataaataatgtggattaagctcctactggaaggttaagtgtttgtacactttcaaacatgcgcaacgcaccaggcaactattgttctgaggagggatgtgatgcggagtaaaatgtggaggaattacggggcttgcagacatatataaactctagccattcggctgaatataagctagaaaccacttcttctctttccatctacatctccctcatctatcaataagaactataatatcaacaatactataatataatatatacacacatttacaaaaaaaatgaagtgtaacgataagatggttgttattatgggtgctactggttctggtaaatcttcattgtcagttgatttggctttacatttcaaagcagaaattattaactctgataagatgcaattctacgatggtttgaagatcactacaaaccaatcaacaattgaagatagaagaggtgttccacatcatttgttgggtgaattaaatccagaagctggtgaagttactgctgcagaattcagagttatggctgcagaagctatctctgaaatcacacaaagaaagaaattgccaatcttggcaggtggttctaactcatacatccatgctttgttagcaaaatcttacgatccagaaaactacccattttcagatcataagggttctatctgttcagaattgaagtacgattgttgtttcatctggatcgatgttgatcaatctgttttatttgaatatttgtcattgagattggatttgatgatgaaatctggcatgttcgaagaaatcgctgaattccatagatctaagaaagcaccaaaagaaccattgggtatttggaaagctattggtgttcaagaattcgatgattacttaaagatgtacaagtgggataatgatatggataaatgggatccaatgagaaaagaagcatacgaaaaagctgttagagcaattaaagaaaatacttttcaattgacaaaggatcaaatcactaaaattaataagttaagaaatgctggttgggatattaagaaagttgatgctacagcatcttttagagaagctattagagctgcaaaagaaggtgaaggtgttgcagaaatgcaaagaaagatctggaataaggaagttttggaaccatgtgttaagatcgttagatctcatttggatcaaccaattaattattactattactatttttatttgttaaaaagatttttgtcattaaattaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaat

SEQ ID NO: 16 - DNA, 2200 bp, synthetic construct
  source 1..2200: mol_type = other DNA
cggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgttgttgactatcttaaaatcattgttagttatttttgttactacaattttgagagttttatatgatactatttcttgttactggttgacaccaagaagaattaagaaaattatggaacaacaaggtgttactggtccaaagccaagaccattaactggtaacatcttggaaatctcagctatggtttctcaatcagcatctaaggattgtgattctatccatcatgatatcgttggtagattgttaccacattatgttgcttggtctaagcaatacggtaaaagattcattgtttggaatggtactgatccaagattgtgtttaactgaaacagaattgattaaagaattgttgatgaagcataatggtgtttcaggtagatcttggttgcagcaacaaggtactaaaaatttcatcggtagaggtttgttgatggcaaatggtcaagattggcatcatcaaagacatttggctgcaccagcttttactggtgaaagattgaaaggttatgcaagacatatggttgaatgtacatcaaagttggttgaaagattgagaaaagaagttggtgaaggtgctaatgaagttgaaatcggtgaagaaatgcataagttgactgcagatatcatctctagaacaaaatttggttcttcattcgaaaagggtaaagaattgtttaatcatttgactgttttacaaagaagatgtgctcaagcaacaagacatttgtgttttccaggttcaagatttttgccatctaagtacaacagagaaattaaatcattgaagaaagaagttgaaagattgttaattgaaattattcaatctagaagagattgtgctgaaatgggtagatcttcaactcatggtgacgatttgttaggtttgttattgaatgaaatggatattgataagaacaataataacaacaacaacaatttgcaattaattatggatgaatgtaaaacatttttctttgctggtcatgaaactacagcattattgttgacttggactacaatgttgttagctgataatccaacatggcaagaaaaagttagagaagaagttagagaagtttttggtagaaatggtttgccatcagttgatcaattgtctaagttgacttcattgtctaaagttattaatgaatcattgagattatatccaccagctactttgttaccaagaatggcatttgaagatttgaaattgggtgacttgacaattccaaaaggtttgtcaatttggattccagttttggctatccatcattctgaagaattgtggggtaaagatgcaaatcaattcaatccagaaagatttggtggtagaccatttgcttctggtagacattttattccatttgctgcaggtccaagaaactgtatcggtcaacaattcgctttgatggaagcaaaaattattttagctactttgatttcaaagtttaatttcacaatctctaaaaattacagacatgcaccaatcgttgttttgacaattaaaccaaagtacggtgttcaagttattttaaaaccattggtttcttaagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaat

SEQ ID NO: 17 - DNA, 2714 bp, synthetic construct
  source 1..2714: mol_type = other DNA
ggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgacttctgctttgtatgcatcagatttgtttaaacaattaaaatctatcatgggtacagattctttgtcagatgatgttgttttagttattgctactacatcattggctttagttgcaggtttcgttgttttgttgtggaagaaaactacagctgatagatctggtgaattgaagccattgatgatcccaaaatcattgatggctaaggatgaagatgatgatttggatttgggttctggtaaaactagagtttcaattttctttggtactcaaacaggtactgctgaaggtttcgctaaggcattgtctgaagaaattaaagcaagatacgaaaaggctgcagttaaagttattgatttggatgattatgctgcagatgatgatcaatacgaagaaaagttgaagaaagaaactttggctttcttttgtgttgcaacttatggtgacggtgaaccaacagataacgctgcaagattctacaagtggttcacagaagaaaacgaaagagatatcaagttgcaacaattagcttatggtgtttttgcattgggtaacagacaatacgaacatttcaataagatcggtatcgttttggatgaagaattatgcaagaaaggtgctaagagattgattgaagttggtttaggtgacgatgatcaatctatcgaagatgatttcaacgcttggaaggaatctttgtggtcagaattggataagttgttgaaggatgaagatgataagtctgttgctacaccatatactgcagttattccagaatacagagttgttactcatgatccaagattcactacacaaaaatctatggaatcaaacgttgctaacggtaacactacaatcgatatccatcatccatgtagagttgatgttgctgttcaaaaggaattgcatacacatgaatctgatagatcatgtatccatttggaattcgatatctctagaacaggtatcacttatgaaacaggtgaccatgttggtgtttacgctgaaaatcatgttgaaattgttgaagaagctggtaaattgttaggtcattctttggatttggttttctctattcatgctgataaagaagatggttctccattagaatcagcagttccaccaccatttccaggtccatgtactttgggtacaggtttagctagatatgcagatttgttaaatccaccaagaaaatctgctttggttgcattagctgcatacgctactgaaccatcagaagcagaaaaattgaaacatttgacatctccagatggtaaagatgaatattcacaatggattgttgcttctcaaagatcattgttagaagttatggctgcatttccatctgctaaaccaccattgggtgttttctttgctgcaattgcaccaagattgcaaccaagatactactcaatctcttcatctccaagattggctccatctagagttcatgttacttcagcattagtttacggtccaacaccaactggtagaattcataaaggtgtttgttctacatggatgaaaaatgctgttccagcagaaaaatctcatgaatgttcaggtgctccaattttcattagagcatcaaacttcaagttgccatctaacccatcaactccaattgttatggttggtccaggtacaggtttagctccttttagaggtttcttgcaagaaagaatggcattaaaagaagatggtgaagaattgggttcatctttgttgtttttcggttgtagaaatagacaaatggatttcatctatgaagatgaattgaacaactttgttgatcaaggtgttatttctgaattgatcatggctttttcaagagaaggtgcacaaaaggaatacgttcaacataagatgatggaaaaagctgcacaagtttgggatttgattaaagaagaaggttatttgtacgtttgtggtgacgctaagggtatggcaagagatgttcatagaactttgcatacaatcgttcaagaacaagaaggtgtttcatcttcagaagctgaagcaattgttaagaaattgcaaactgaaggtagatatttgagagatgtttggtaagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttc

SEQ ID NO: 18 - DNA, 1385 bp, synthetic construct
  source 1..1385: mol_type = other DNA
ggatccccgggtacccagttgatcaaaaatcatcgcttcgctgattaattaccccagaaataaggctaaaaaactaatcgcattatcatcctatggttgttaatttgattcgttaatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtaatgccatgtaatatgattattaaacttctttgcgtccatccaaaaaaaaagtaagaatttttgaaaattcaatataaaaaaaaatggaagaaactaaatcaagattcaaaagaatctgtgttttctgtggttcttcatctggtaaaaagccatcttatcaagaagctgcaatccaattgggtaacgaattggttgaaagaagaatcgatttggtttacggtggtggttcagttggtttgatgggtttagtttctcaagctgttcatcatggtggtagacatgttttgggtgttattccaaagacattgatgccaagagaaattactggtgaaacaattggtgaagttaaagctgttgcagatatgcatcaaagaaaagctgaaatggcaagacaagctgatgcttttattgctttgccaggtggttatggtactttggaagaattgttggaagttattacatgggcacaattgggtatccatagaaagccagttggtttgttgaacgttgatggttactacaactcattgttgacttttattgataaggctgttgatgaaggttttatttcaccaatggcaagaagaatcattgtttctgctccaaacgcaaaggaattggttagacaattggaagaatacgaaccagaattcgatgaaatcacatctaagttggtttgggatgaagttgatagaatttcatacgttccaggttctgaagttgctactgcaacataagcttatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 19 - DNA, 2524 bp, synthetic construct
  source 1..2524: mol_type = other DNA
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatcaatggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaat

SEQ ID NO: 20 - DNA, 2807 bp, synthetic construct
  source 1..2807: mol_type = other DNA
ccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcatcaatttctcaagtttcagtttcatttttcttgttctattacaactttttttacttcttgctcattagaaagaaagcatagcaatctaatctaagttttaattacaaaagatcaatgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctgagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttc

SEQ ID NO: 21 - DNA, 2054 bp, synthetic construct
  source 1..2054: mol_type = other DNA
gaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaaaaaaaatgatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaat

SEQ ID NO: 22 - DNA, 2544 bp, synthetic construct
  source 1..2544: mol_type = other DNA
ccacagagaacaggagattacaagatccagatcaccaaaatgccggaacacaggctgaaaaactccaagcagccgcacacccgatttttagccagcagattcatacctaactccaatgaattgcagcatatatcctgcaataacacattaaggatcgggccttggacggccgaatatatgattatagtccttttgtcgccaaaaagtcgcctcgaggtcacgtgcatccgaaatcaccccctgagcacttcccgcccagaaaacagggaaggggcggcaccagcaggcaccggcattcactcgtccgtgcggcgtggtctgtgccgttcaggggcctgcgtgcctgtgatgatatcgctgcagggcaccgagtcggagaggaccactccgagccgaggttagttaagccattctctacgctaaaaaataaataatgtggattaagctcctactggaaggttaagtgtttgtacactttcaaacatgcgcaacgcaccaggcaactattgttctgaggagggatgtgatgcggagtaaaatgtggaggaattacggggcttgcagacatatataaactctagccattcggctgaatataagctagaaaccacttcttctctttccatctacatctccctcatctatcaataagaactataatatcaacaatactataatataatatatacacacatttacaaaaaaaatggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaat

SEQ ID NO: 23 - DNA, 2507 bp, synthetic construct
  source 1..2507: mol_type = other DNA
ggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctgagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttc

SEQ ID NO: 24 - DNA, 1513 bp, synthetic construct
  source 1..1513: mol_type = other DNA
cggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaat

SEQ ID NO: 25 - DNA, 8255 bp, synthetic construct
  source 1..8255: mol_type = other DNA
ccacagagaacaggagattacaagatccagatcaccaaaatgccggaacacaggctgaaaaactccaagcagccgcacacccgatttttagccagcagattcatacctaactccaatgaattgcagcatatatcctgcaataacacattaaggatcgggccttggacggccgaatatatgattatagtccttttgtcgccaaaaagtcgcctcgaggtcacgtgcatccgaaatcaccccctgagcacttcccgcccagaaaacagggaaggggcggcaccagcaggcaccggcattcactcgtccgtgcggcgtggtctgtgccgttcaggggcctgcgtgcctgtgatgatatcgctgcagggcaccgagtcggagaggaccactccgagccgaggttagttaagccattctctacgctaaaaaataaataatgtggattaagctcctactggaaggttaagtgtttgtacactttcaaacatgcgcaacgcaccaggcaactattgttctgaggagggatgtgatgcggagtaaaatgtggaggaattacggggcttgcagacatatataaactctagccattcggctgaatataagctagaaaccacttcttctctttccatctacatctccctcatctatcaataagaactataatatcaacaatactataatataatatatacacacatttacaaaaaaaatgaagtgtaacgataagatggttgttattatgggtgctactggttctggtaaatcttcattgtcagttgatttggctttacatttcaaagcagaaattattaactctgataagatgcaattctacgatggtttgaagatcactacaaaccaatcaacaattgaagatagaagaggtgttccacatcatttgttgggtgaattaaatccagaagctggtgaagttactgctgcagaattcagagttatggctgcagaagctatctctgaaatcacacaaagaaagaaattgccaatcttggcaggtggttctaactcatacatccatgctttgttagcaaaatcttacgatccagaaaactacccattttcagatcataagggttctatctgttcagaattgaagtacgattgttgtttcatctggatcgatgttgatcaatctgttttatttgaatatttgtcattgagattggatttgatgatgaaatctggcatgttcgaagaaatcgctgaattccatagatctaagaaagcaccaaaagaaccattgggtatttggaaagctattggtgttcaagaattcgatgattacttaaagatgtacaagtgggataatgatatggataaatgggatccaatgagaaaagaagcatacgaaaaagctgttagagcaattaaagaaaatacttttcaattgacaaaggatcaaatcactaaaattaataagttaagaaatgctggttgggatattaagaaagttgatgctacagcatcttttagagaagctattagagctgcaaaagaaggtgaaggtgttgcagaaatgcaaagaaagatctggaataaggaagttttggaaccatgtgttaagatcgttagatctcatttggatcaaccaattaattattactattactatttttatttgttaaaaagatttttgtcattaaattaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatcgctagcaaggagcggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgttgttgactatcttaaaatcattgttagttatttttgttactacaattttgagagttttatatgatactatttcttgttactggttgacaccaagaagaattaagaaaattatggaacaacaaggtgttactggtccaaagccaagaccattaactggtaacatcttggaaatctcagctatggtttctcaatcagcatctaaggattgtgattctatccatcatgatatcgttggtagattgttaccacattatgttgcttggtctaagcaatacggtaaaagattcattgtttggaatggtactgatccaagattgtgtttaactgaaacagaattgattaaagaattgttgatgaagcataatggtgtttcaggtagatcttggttgcagcaacaaggtactaaaaatttcatcggtagaggtttgttgatggcaaatggtcaagattggcatcatcaaagacatttggctgcaccagcttttactggtgaaagattgaaaggttatgcaagacatatggttgaatgtacatcaaagttggttgaaagattgagaaaagaagttggtgaaggtgctaatgaagttgaaatcggtgaagaaatgcataagttgactgcagatatcatctctagaacaaaatttggttcttcattcgaaaagggtaaagaattgtttaatcatttgactgttttacaaagaagatgtgctcaagcaacaagacatttgtgttttccaggttcaagatttttgccatctaagtacaacagagaaattaaatcattgaagaaagaagttgaaagattgttaattgaaattattcaatctagaagagattgtgctgaaatgggtagatcttcaactcatggtgacgatttgttaggtttgttattgaatgaaatggatattgataagaacaataataacaacaacaacaatttgcaattaattatggatgaatgtaaaacatttttctttgctggtcatgaaactacagcattattgttgacttggactacaatgttgttagctgataatccaacatggcaagaaaaagttagagaagaagttagagaagtttttggtagaaatggtttgccatcagttgatcaattgtctaagttgacttcattgtctaaagttattaatgaatcattgagattatatccaccagctactttgttaccaagaatggcatttgaagatttgaaattgggtgacttgacaattccaaaaggtttgtcaatttggattccagttttggctatccatcattctgaagaattgtggggtaaagatgcaaatcaattcaatccagaaagatttggtggtagaccatttgcttctggtagacattttattccatttgctgcaggtccaagaaactgtatcggtcaacaattcgctttgatggaagcaaaaattattttagctactttgatttcaaagtttaatttcacaatctctaaaaattacagacatgcaccaatcgttgttttgacaattaaaccaaagtacggtgttcaagttattttaaaaccattggtttcttaagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctatacaggagggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgacttctgctttgtatgcatcagatttgtttaaacaattaaaatctatcatgggtacagattctttgtcagatgatgttgttttagttattgctactacatcattggctttagttgcaggtttcgttgttttgttgtggaagaaaactacagctgatagatctggtgaattgaagccattgatgatcccaaaatcattgatggctaaggatgaagatgatgatttggatttgggttctggtaaaactagagtttcaattttctttggtactcaaacaggtactgctgaaggtttcgctaaggcattgtctgaagaaattaaagcaagatacgaaaaggctgcagttaaagttattgatttggatgattatgctgcagatgatgatcaatacgaagaaaagttgaagaaagaaactttggctttcttttgtgttgcaacttatggtgacggtgaaccaacagataacgctgcaagattctacaagtggttcacagaagaaaacgaaagagatatcaagttgcaacaattagcttatggtgtttttgcattgggtaacagacaatacgaacatttcaataagatcggtatcgttttggatgaagaattatgcaagaaaggtgctaagagattgattgaagttggtttaggtgacgatgatcaatctatcgaagatgatttcaacgcttggaaggaatctttgtggtcagaattggataagttgttgaaggatgaagatgataagtctgttgctacaccatatactgcagttattccagaatacagagttgttactcatgatccaagattcactacacaaaaatctatggaatcaaacgttgctaacggtaacactacaatcgatatccatcatccatgtagagttgatgttgctgttcaaaaggaattgcatacacatgaatctgatagatcatgtatccatttggaattcgatatctctagaacaggtatcacttatgaaacaggtgaccatgttggtgtttacgctgaaaatcatgttgaaattgttgaagaagctggtaaattgttaggtcattctttggatttggttttctctattcatgctgataaagaagatggttctccattagaatcagcagttccaccaccatttccaggtccatgtactttgggtacaggtttagctagatatgcagatttgttaaatccaccaagaaaatctgctttggttgcattagctgcatacgctactgaaccatcagaagcagaaaaattgaaacatttgacatctccagatggtaaagatgaatattcacaatggattgttgcttctcaaagatcattgttagaagttatggctgcatttccatctgctaaaccaccattgggtgttttctttgctgcaattgcaccaagattgcaaccaagatactactcaatctcttcatctccaagattggctccatctagagttcatgttacttcagcattagtttacggtccaacaccaactggtagaattcataaaggtgtttgttctacatggatgaaaaatgctgttccagcagaaaaatctcatgaatgttcaggtgctccaattttcattagagcatcaaacttcaagttgccatctaacccatcaactccaattgttatggttggtccaggtacaggtttagctccttttagaggtttcttgcaagaaagaatggcattaaaagaagatggtgaagaattgggttcatctttgttgtttttcggttgtagaaatagacaaatggatttcatctatgaagatgaattgaacaactttgttgatcaaggtgttatttctgaattgatcatggctttttcaagagaaggtgcacaaaaggaatacgttcaacataagatgatggaaaaagctgcacaagtttgggatttgattaaagaagaaggttatttgtacgtttgtggtgacgctaagggtatggcaagagatgttcatagaactttgcatacaatcgttcaagaacaagaaggtgtttcatcttcagaagctgaagcaattgttaagaaattgcaaactgaaggtagatatttgagagatgtttggtaagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttccgctacagaggagggatccccgggtacccagttgatcaaaaatcatcgcttcgctgattaattaccccagaaataaggctaaaaaactaatcgcattatcatcctatggttgttaatttgattcgttaatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtaatgccatgtaatatgattattaaacttctttgcgtccatccaaaaaaaaagtaagaatttttgaaaattcaatataaaaaaaaatggaagaaactaaatcaagattcaaaagaatctgtgttttctgtggttcttcatctggtaaaaagccatcttatcaagaagctgcaatccaattgggtaacgaattggttgaaagaagaatcgatttggtttacggtggtggttcagttggtttgatgggtttagtttctcaagctgttcatcatggtggtagacatgttttgggtgttattccaaagacattgatgccaagagaaattactggtgaaacaattggtgaagttaaagctgttgcagatatgcatcaaagaaaagctgaaatggcaagacaagctgatgcttttattgctttgccaggtggttatggtactttggaagaattgttggaagttattacatgggcacaattgggtatccatagaaagccagttggtttgttgaacgttgatggttactacaactcattgttgacttttattgataaggctgttgatgaaggttttatttcaccaatggcaagaagaatcattgtttctgctccaaacgcaaaggaattggttagacaattggaagaatacgaaccagaattcgatgaaatcacatctaagttggtttgggatgaagttgatagaatttcatacgttccaggttctgaagttgctactgcaacataagcttatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 26 - DNA, 6325 bp, synthetic construct
  source 1..6325: mol_type = other DNA
cggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgttgttgactatcttaaaatcattgttagttatttttgttactacaattttgagagttttatatgatactatttcttgttactggttgacaccaagaagaattaagaaaattatggaacaacaaggtgttactggtccaaagccaagaccattaactggtaacatcttggaaatctcagctatggtttctcaatcagcatctaaggattgtgattctatccatcatgatatcgttggtagattgttaccacattatgttgcttggtctaagcaatacggtaaaagattcattgtttggaatggtactgatccaagattgtgtttaactgaaacagaattgattaaagaattgttgatgaagcataatggtgtttcaggtagatcttggttgcagcaacaaggtactaaaaatttcatcggtagaggtttgttgatggcaaatggtcaagattggcatcatcaaagacatttggctgcaccagcttttactggtgaaagattgaaaggttatgcaagacatatggttgaatgtacatcaaagttggttgaaagattgagaaaagaagttggtgaaggtgctaatgaagttgaaatcggtgaagaaatgcataagttgactgcagatatcatctctagaacaaaatttggttcttcattcgaaaagggtaaagaattgtttaatcatttgactgttttacaaagaagatgtgctcaagcaacaagacatttgtgttttccaggttcaagatttttgccatctaagtacaacagagaaattaaatcattgaagaaagaagttgaaagattgttaattgaaattattcaatctagaagagattgtgctgaaatgggtagatcttcaactcatggtgacgatttgttaggtttgttattgaatgaaatggatattgataagaacaataataacaacaacaacaatttgcaattaattatggatgaatgtaaaacatttttctttgctggtcatgaaactacagcattattgttgacttggactacaatgttgttagctgataatccaacatggcaagaaaaagttagagaagaagttagagaagtttttggtagaaatggtttgccatcagttgatcaattgtctaagttgacttcattgtctaaagttattaatgaatcattgagattatatccaccagctactttgttaccaagaatggcatttgaagatttgaaattgggtgacttgacaattccaaaaggtttgtcaatttggattccagttttggctatccatcattctgaagaattgtggggtaaagatgcaaatcaattcaatccagaaagatttggtggtagaccatttgcttctggtagacattttattccatttgctgcaggtccaagaaactgtatcggtcaacaattcgctttgatggaagcaaaaattattttagctactttgatttcaaagtttaatttcacaatctctaaaaattacagacatgcaccaatcgttgttttgacaattaaaccaaagtacggtgttcaagttattttaaaaccattggtttcttaagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctatacaggagggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgacttctgctttgtatgcatcagatttgtttaaacaattaaaatctatcatgggtacagattctttgtcagatgatgttgttttagttattgctactacatcattggctttagttgcaggtttcgttgttttgttgtggaagaaaactacagctgatagatctggtgaattgaagccattgatgatcccaaaatcattgatggctaaggatgaagatgatgatttggatttgggttctggtaaaactagagtttcaattttctttggtactcaaacaggtactgctgaaggtttcgctaaggcattgtctgaagaaattaaagcaagatacgaaaaggctgcagttaaagttattgatttggatgattatgctgcagatgatgatcaatacgaagaaaagttgaagaaagaaactttggctttcttttgtgttgcaacttatggtgacggtgaaccaacagataacgctgcaagattctacaagtggttcacagaagaaaacgaaagagatatcaagttgcaacaattagcttatggtgtttttgcattgggtaacagacaatacgaacatttcaataagatcggtatcgttttggatgaagaattatgcaagaaaggtgctaagagattgattgaagttggtttaggtgacgatgatcaatctatcgaagatgatttcaacgcttggaaggaatctttgtggtcagaattggataagttgttgaaggatgaagatgataagtctgttgctacaccatatactgcagttattccagaatacagagttgttactcatgatccaagattcactacacaaaaatctatggaatcaaacgttgctaacggtaacactacaatcgatatccatcatccatgtagagttgatgttgctgttcaaaaggaattgcatacacatgaatctgatagatcatgtatccatttggaattcgatatctctagaacaggtatcacttatgaaacaggtgaccatgttggtgtttacgctgaaaatcatgttgaaattgttgaagaagctggtaaattgttaggtcattctttggatttggttttctctattcatgctgataaagaagatggttctccattagaatcagcagttccaccaccatttccaggtccatgtactttgggtacaggtttagctagatatgcagatttgttaaatccaccaagaaaatctgctttggttgcattagctgcatacgctactgaaccatcagaagcagaaaaattgaaacatttgacatctccagatggtaaagatgaatattcacaatggattgttgcttctcaaagatcattgttagaagttatggctgcatttccatctgctaaaccaccattgggtgttttctttgctgcaattgcaccaagattgcaaccaagatactactcaatctcttcatctccaagattggctccatctagagttcatgttacttcagcattagtttacggtccaacaccaactggtagaattcataaaggtgtttgttctacatggatgaaaaatgctgttccagcagaaaaatctcatgaatgttcaggtgctccaattttcattagagcatcaaacttcaagttgccatctaacccatcaactccaattgttatggttggtccaggtacaggtttagctccttttagaggtttcttgcaagaaagaatggcattaaaagaagatggtgaagaattgggttcatctttgttgtttttcggttgtagaaatagacaaatggatttcatctatgaagatgaattgaacaactttgttgatcaaggtgttatttctgaattgatcatggctttttcaagagaaggtgcacaaaaggaatacgttcaacataagatgatggaaaaagctgcacaagtttgggatttgattaaagaagaaggttatttgtacgtttgtggtgacgctaagggtatggcaagagatgttcatagaactttgcatacaatcgttcaagaacaagaaggtgtttcatcttcagaagctgaagcaattgttaagaaattgcaaactgaaggtagatatttgagagatgtttggtaagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttccgctacagaggagggatccccgggtacccagttgatcaaaaatcatcgcttcgctgattaattaccccagaaataaggctaaaaaactaatcgcattatcatcctatggttgttaatttgattcgttaatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtaatgccatgtaatatgattattaaacttctttgcgtccatccaaaaaaaaagtaagaatttttgaaaattcaatataaaaaaaaatggaagaaactaaatcaagattcaaaagaatctgtgttttctgtggttcttcatctggtaaaaagccatcttatcaagaagctgcaatccaattgggtaacgaattggttgaaagaagaatcgatttggtttacggtggtggttcagttggtttgatgggtttagtttctcaagctgttcatcatggtggtagacatgttttgggtgttattccaaagacattgatgccaagagaaattactggtgaaacaattggtgaagttaaagctgttgcagatatgcatcaaagaaaagctgaaatggcaagacaagctgatgcttttattgctttgccaggtggttatggtactttggaagaattgttggaagttattacatgggcacaattgggtatccatagaaagccagttggtttgttgaacgttgatggttactacaactcattgttgacttttattgataaggctgttgatgaaggttttatttcaccaatggcaagaagaatcattgtttctgctccaaacgcaaaggaattggttagacaattggaagaatacgaaccagaattcgatgaaatcacatctaagttggtttgggatgaagttgatagaatttcatacgttccaggttctgaagttgctactgcaacataagcttatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 27 - DNA, 7422 bp, synthetic construct
  source 1..7422: mol_type = other DNA
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatcaatggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatcgctatacgctcttcatacaggaggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaaaaaaaatgatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctacagaggagccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcatcaatttctcaagtttcagtttcatttttcttgttctattacaactttttttacttcttgctcattagaaagaaagcatagcaatctaatctaagttttaattacaaaagatcaatgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctgagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttc

SEQ ID NO: 28 - DNA, 6590 bp, synthetic construct
  source 1..6590: mol_type = other DNA
ccacagagaacaggagattacaagatccagatcaccaaaatgccggaacacaggctgaaaaactccaagcagccgcacacccgatttttagccagcagattcatacctaactccaatgaattgcagcatatatcctgcaataacacattaaggatcgggccttggacggccgaatatatgattatagtccttttgtcgccaaaaagtcgcctcgaggtcacgtgcatccgaaatcaccccctgagcacttcccgcccagaaaacagggaaggggcggcaccagcaggcaccggcattcactcgtccgtgcggcgtggtctgtgccgttcaggggcctgcgtgcctgtgatgatatcgctgcagggcaccgagtcggagaggaccactccgagccgaggttagttaagccattctctacgctaaaaaataaataatgtggattaagctcctactggaaggttaagtgtttgtacactttcaaacatgcgcaacgcaccaggcaactattgttctgaggagggatgtgatgcggagtaaaatgtggaggaattacggggcttgcagacatatataaactctagccattcggctgaatataagctagaaaccacttcttctctttccatctacatctccctcatctatcaataagaactataatatcaacaatactataatataatatatacacacatttacaaaaaaaatggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatcgctatacaggagcggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctacagaggagggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctgagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttc

SEQ ID NO: 29 - DNA, 9481 bp, synthetic construct
  source 1..9481: mol_type = other DNA
ggccgcgcggagaagtcgttgatagcatttccgaaggcttttccacattgactgcagtatctaaaagagctccagcagcggtcttagtagttcctgtgcttgccgctttgcagtatacagggacattgggaactacggaggaaaaatcggaatgctctgagcccttctcaaaaccagacgaactcgtcttatttctcagtaaattacccaacatataaactctccctcttcaccttctctacaaggccacacacaagacagttttaacaggcaaatggtactggaggctctcaccaatggggtttaagacgctccaaacacacgctctcctttttttctttatatatcggtagcaacgaaagctagtcgcagaacgttacacactaaagcagatcaatcaatcaatcctgagaacctgcttcgttcaaaccatccactagaagaaacacgcacgtgtacccaaaaatagagacaaacaaacagacaaaaataacaatgattcctaccgactaaagtaaaacaacactcacgtaataaaaaatcctagatacaactcacttgtattcctctcttctttgaccatcactaaagcttctctcttactatattcctcaaataaaaaaaagttgcaatgattacttaccaatgtgccataaactccgtgcaccacgctaggtatactaatgaggagccacagagaacaggagattacaagatccagatcaccaaaatgccggaacacaggctgaaaaactccaagcagccgcacacccgatttttagccagcagattcatacctaactccaatgaattgcagcatatatcctgcaataacacattaaggatcgggccttggacggccgaatatatgattatagtccttttgtcgccaaaaagtcgcctcgaggtcacgtgcatccgaaatcaccccctgagcacttcccgcccagaaaacagggaaggggcggcaccagcaggcaccggcattcactcgtccgtgcggcgtggtctgtgccgttcaggggcctgcgtgcctgtgatgatatcgctgcagggcaccgagtcggagaggaccactccgagccgaggttagttaagccattctctacgctaaaaaataaataatgtggattaagctcctactggaaggttaagtgtttgtacactttcaaacatgcgcaacgcaccaggcaactattgttctgaggagggatgtgatgcggagtaaaatgtggaggaattacggggcttgcagacatatataaactctagccattcggctgaatataagctagaaaccacttcttctctttccatctacatctccctcatctatcaataagaactataatatcaacaatactataatataatatatacacacatttacaaaaaaaatgaagtgtaacgataagatggttgttattatgggtgctactggttctggtaaatcttcattgtcagttgatttggctttacatttcaaagcagaaattattaactctgataagatgcaattctacgatggtttgaagatcactacaaaccaatcaacaattgaagatagaagaggtgttccacatcatttgttgggtgaattaaatccagaagctggtgaagttactgctgcagaattcagagttatggctgcagaagctatctctgaaatcacacaaagaaagaaattgccaatcttggcaggtggttctaactcatacatccatgctttgttagcaaaatcttacgatccagaaaactacccattttcagatcataagggttctatctgttcagaattgaagtacgattgttgtttcatctggatcgatgttgatcaatctgttttatttgaatatttgtcattgagattggatttgatgatgaaatctggcatgttcgaagaaatcgctgaattccatagatctaagaaagcaccaaaagaaccattgggtatttggaaagctattggtgttcaagaattcgatgattacttaaagatgtacaagtgggataatgatatggataaatgggatccaatgagaaaagaagcatacgaaaaagctgttagagcaattaaagaaaatacttttcaattgacaaaggatcaaatcactaaaattaataagttaagaaatgctggttgggatattaagaaagttgatgctacagcatcttttagagaagctattagagctgcaaaagaaggtgaaggtgttgcagaaatgcaaagaaagatctggaataaggaagttttggaaccatgtgttaagatcgttagatctcatttggatcaaccaattaattattactattactatttttatttgttaaaaagatttttgtcattaaattaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatcgctagcaaggagcggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgttgttgactatcttaaaatcattgttagttatttttgttactacaattttgagagttttatatgatactatttcttgttactggttgacaccaagaagaattaagaaaattatggaacaacaaggtgttactggtccaaagccaagaccattaactggtaacatcttggaaatctcagctatggtttctcaatcagcatctaaggattgtgattctatccatcatgatatcgttggtagattgttaccacattatgttgcttggtctaagcaatacggtaaaagattcattgtttggaatggtactgatccaagattgtgtttaactgaaacagaattgattaaagaattgttgatgaagcataatggtgtttcaggtagatcttggttgcagcaacaaggtactaaaaatttcatcggtagaggtttgttgatggcaaatggtcaagattggcatcatcaaagacatttggctgcaccagcttttactggtgaaagattgaaaggttatgcaagacatatggttgaatgtacatcaaagttggttgaaagattgagaaaagaagttggtgaaggtgctaatgaagttgaaatcggtgaagaaatgcataagttgactgcagatatcatctctagaacaaaatttggttcttcattcgaaaagggtaaagaattgtttaatcatttgactgttttacaaagaagatgtgctcaagcaacaagacatttgtgttttccaggttcaagatttttgccatctaagtacaacagagaaattaaatcattgaagaaagaagttgaaagattgttaattgaaattattcaatctagaagagattgtgctgaaatgggtagatcttcaactcatggtgacgatttgttaggtttgttattgaatgaaatggatattgataagaacaataataacaacaacaacaatttgcaattaattatggatgaatgtaaaacatttttctttgctggtcatgaaactacagcattattgttgacttggactacaatgttgttagctgataatccaacatggcaagaaaaagttagagaagaagttagagaagtttttggtagaaatggtttgccatcagttgatcaattgtctaagttgacttcattgtctaaagttattaatgaatcattgagattatatccaccagctactttgttaccaagaatggcatttgaagatttgaaattgggtgacttgacaattccaaaaggtttgtcaatttggattccagttttggctatccatcattctgaagaattgtggggtaaagatgcaaatcaattcaatccagaaagatttggtggtagaccatttgcttctggtagacattttattccatttgctgcaggtccaagaaactgtatcggtcaacaattcgctttgatggaagcaaaaattattttagctactttgatttcaaagtttaatttcacaatctctaaaaattacagacatgcaccaatcgttgttttgacaattaaaccaaagtacggtgttcaagttattttaaaaccattggtttcttaagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctatacaggagggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgacttctgctttgtatgcatcagatttgtttaaacaattaaaatctatcatgggtacagattctttgtcagatgatgttgttttagttattgctactacatcattggctttagttgcaggtttcgttgttttgttgtggaagaaaactacagctgatagatctggtgaattgaagccattgatgatcccaaaatcattgatggctaaggatgaagatgatgatttggatttgggttctggtaaaactagagtttcaattttctttggtactcaaacaggtactgctgaaggtttcgctaaggcattgtctgaagaaattaaagcaagatacgaaaaggctgcagttaaagttattgatttggatgattatgctgcagatgatgatcaatacgaagaaaagttgaagaaagaaactttggctttcttttgtgttgcaacttatggtgacggtgaaccaacagataacgctgcaagattctacaagtggttcacagaagaaaacgaaagagatatcaagttgcaacaattagcttatggtgtttttgcattgggtaacagacaatacgaacatttcaataagatcggtatcgttttggatgaagaattatgcaagaaaggtgctaagagattgattgaagttggtttaggtgacgatgatcaatctatcgaagatgatttcaacgcttggaaggaatctttgtggtcagaattggataagttgttgaaggatgaagatgataagtctgttgctacaccatatactgcagttattccagaatacagagttgttactcatgatccaagattcactacacaaaaatctatggaatcaaacgttgctaacggtaacactacaatcgatatccatcatccatgtagagttgatgttgctgttcaaaaggaattgcatacacatgaatctgatagatcatgtatccatttggaattcgatatctctagaacaggtatcacttatgaaacaggtgaccatgttggtgtttacgctgaaaatcatgttgaaattgttgaagaagctggtaaattgttaggtcattctttggatttggttttctctattcatgctgataaagaagatggttctccattagaatcagcagttccaccaccatttccaggtccatgtactttgggtacaggtttagctagatatgcagatttgttaaatccaccaagaaaatctgctttggttgcattagctgcatacgctactgaaccatcagaagcagaaaaattgaaacatttgacatctccagatggtaaagatgaatattcacaatggattgttgcttctcaaagatcattgttagaagttatggctgcatttccatctgctaaaccaccattgggtgttttctttgctgcaattgcaccaagattgcaaccaagatactactcaatctcttcatctccaagattggctccatctagagttcatgttacttcagcattagtttacggtccaacaccaactggtagaattcataaaggtgtttgttctacatggatgaaaaatgctgttccagcagaaaaatctcatgaatgttcaggtgctccaattttcattagagcatcaaacttcaagttgccatctaacccatcaactccaattgttatggttggtccaggtacaggtttagctccttttagaggtttcttgcaagaaagaatggcattaaaagaagatggtgaagaattgggttcatctttgttgtttttcggttgtagaaatagacaaatggatttcatctatgaagatgaattgaacaactttgttgatcaaggtgttatttctgaattgatcatggctttttcaagagaaggtgcacaaaaggaatacgttcaacataagatgatggaaaaagctgcacaagtttgggatttgattaaagaagaaggttatttgtacgtttgtggtgacgctaagggtatggcaagagatgttcatagaactttgcatacaatcgttcaagaacaagaaggtgtttcatcttcagaagctgaagcaattgttaagaaattgcaaactgaaggtagatatttgagagatgtttggtaagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttccgctacagaggagggatccccgggtacccagttgatcaaaaatcatcgcttcgctgattaattaccccagaaataaggctaaaaaactaatcgcattatcatcctatggttgttaatttgattcgttaatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtaatgccatgtaatatgattattaaacttctttgcgtccatccaaaaaaaaagtaagaatttttgaaaattcaatataaaaaaaaatggaagaaactaaatcaagattcaaaagaatctgtgttttctgtggttcttcatctggtaaaaagccatcttatcaagaagctgcaatccaattgggtaacgaattggttgaaagaagaatcgatttggtttacggtggtggttcagttggtttgatgggtttagtttctcaagctgttcatcatggtggtagacatgttttgggtgttattccaaagacattgatgccaagagaaattactggtgaaacaattggtgaagttaaagctgttgcagatatgcatcaaagaaaagctgaaatggcaagacaagctgatgcttttattgctttgccaggtggttatggtactttggaagaattgttggaagttattacatgggcacaattgggtatccatagaaagccagttggtttgttgaacgttgatggttactacaactcattgttgacttttattgataaggctgttgatgaaggttttatttcaccaatggcaagaagaatcattgtttctgctccaaacgcaaaggaattggttagacaattggaagaatacgaaccagaattcgatgaaatcacatctaagttggtttgggatgaagttgatagaatttcatacgttccaggttctgaagttgctactgcaacataagcttatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagcgctaggtaaatgaatgaggaggccttcgatttgacacatctctaagctgaaactgagaatactgttgtaaaacaggtattggctgcttcatagtacacccaattgcccacaacccaagttaatcataagctcttgttgtcgatgaattgctcaaaatgtggccattctttcaattttgcacctctaaaactgctccgtgggttgtgacaacaaccttaagggtaatggacttttttagaaagacaaatgagatgatttcacctactggtatatcatttatttatacacgaccgttttgacatttcagagttgcacgttggtatatcctaatattctcacaagcatcacagtgaagaaacgcaagaaaatggaaaaaattcccaggtggttgttgttttctcttatatcgtcggtgctgtgcatacttggggccctgtgtgtgccgttgttatcggttgccttcgatagcaagcgcaatagccaatctaagttggtcaactatggtctttctctaagtgccggatctatgatcgc

SEQ ID NO: 30 - DNA, 7701 bp, synthetic construct
  source 1..7701: mol_type = other DNA
ggccgcagttacttgctctatgcgtttgcgcatcctctttttactttttttttttcagtaaagcctaagcataaatcgttttatacgtacgacacgttcaacttttcttggttagtagtggcaatctctgcaatacatacagggagtcatggtctatcatcttgtccaatcaaagaagcatcggttcagatcgagcaaactgtagggagaaaggaaagtagaaatgcagagtgtgctatatgtccaatctcggttttgtagtttggatgtcattagagatctaccacccaaccggctgctttcatgtggaacagaaaagaaatcggggcgcttcctcttctgtattcctttaattaacgtttttattcagccatctaaccatcatacccccatacggtaacaaaacctcttctaagaaaagaagtctctgctcctccgccatcttatttttattcgctgcgcgcgtttattgtcgcatcgctagccagcaaaaagttggttgcctttttttacctaaaaaagacacatctaactgattagttttccgttttaggatattgacgccaagcgtgcgtctgattcgctaggtatactaatgaggaggggaacaaaggattgtgcagtgccatacgacaagttcgcctcaatcgccaaaccccttgactgtgcttcccgagcggaaacatttagattggctactacaaccgttgctctccgggcggataagagaataaatctggattcggcagagtagtcggccttatcatcagttagatgttgataatcccgttgggcaggtcacacgctagcaaggagcggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgttgttgactatcttaaaatcattgttagttatttttgttactacaattttgagagttttatatgatactatttcttgttactggttgacaccaagaagaattaagaaaattatggaacaacaaggtgttactggtccaaagccaagaccattaactggtaacatcttggaaatctcagctatggtttctcaatcagcatctaaggattgtgattctatccatcatgatatcgttggtagattgttaccacattatgttgcttggtctaagcaatacggtaaaagattcattgtttggaatggtactgatccaagattgtgtttaactgaaacagaattgattaaagaattgttgatgaagcataatggtgtttcaggtagatcttggttgcagcaacaaggtactaaaaatttcatcggtagaggtttgttgatggcaaatggtcaagattggcatcatcaaagacatttggctgcaccagcttttactggtgaaagattgaaaggttatgcaagacatatggttgaatgtacatcaaagttggttgaaagattgagaaaagaagttggtgaaggtgctaatgaagttgaaatcggtgaagaaatgcataagttgactgcagatatcatctctagaacaaaatttggttcttcattcgaaaagggtaaagaattgtttaatcatttgactgttttacaaagaagatgtgctcaagcaacaagacatttgtgttttccaggttcaagatttttgccatctaagtacaacagagaaattaaatcattgaagaaagaagttgaaagattgttaattgaaattattcaatctagaagagattgtgctgaaatgggtagatcttcaactcatggtgacgatttgttaggtttgttattgaatgaaatggatattgataagaacaataataacaacaacaacaatttgcaattaattatggatgaatgtaaaacatttttctttgctggtcatgaaactacagcattattgttgacttggactacaatgttgttagctgataatccaacatggcaagaaaaagttagagaagaagttagagaagtttttggtagaaatggtttgccatcagttgatcaattgtctaagttgacttcattgtctaaagttattaatgaatcattgagattatatccaccagctactttgttaccaagaatggcatttgaagatttgaaattgggtgacttgacaattccaaaaggtttgtcaatttggattccagttttggctatccatcattctgaagaattgtggggtaaagatgcaaatcaattcaatccagaaagatttggtggtagaccatttgcttctggtagacattttattccatttgctgcaggtccaagaaactgtatcggtcaacaattcgctttgatggaagcaaaaattattttagctactttgatttcaaagtttaatttcacaatctctaaaaattacagacatgcaccaatcgttgttttgacaattaaaccaaagtacggtgttcaagttattttaaaaccattggtttcttaagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctatacaggagggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgacttctgctttgtatgcatcagatttgtttaaacaattaaaatctatcatgggtacagattctttgtcagatgatgttgttttagttattgctactacatcattggctttagttgcaggtttcgttgttttgttgtggaagaaaactacagctgatagatctggtgaattgaagccattgatgatcccaaaatcattgatggctaaggatgaagatgatgatttggatttgggttctggtaaaactagagtttcaattttctttggtactcaaacaggtactgctgaaggtttcgctaaggcattgtctgaagaaattaaagcaagatacgaaaaggctgcagttaaagttattgatttggatgattatgctgcagatgatgatcaatacgaagaaaagttgaagaaagaaactttggctttcttttgtgttgcaacttatggtgacggtgaaccaacagataacgctgcaagattctacaagtggttcacagaagaaaacgaaagagatatcaagttgcaacaattagcttatggtgtttttgcattgggtaacagacaatacgaacatttcaataagatcggtatcgttttggatgaagaattatgcaagaaaggtgctaagagattgattgaagttggtttaggtgacgatgatcaatctatcgaagatgatttcaacgcttggaaggaatctttgtggtcagaattggataagttgttgaaggatgaagatgataagtctgttgctacaccatatactgcagttattccagaatacagagttgttactcatgatccaagattcactacacaaaaatctatggaatcaaacgttgctaacggtaacactacaatcgatatccatcatccatgtagagttgatgttgctgttcaaaaggaattgcatacacatgaatctgatagatcatgtatccatttggaattcgatatctctagaacaggtatcacttatgaaacaggtgaccatgttggtgtttacgctgaaaatcatgttgaaattgttgaagaagctggtaaattgttaggtcattctttggatttggttttctctattcatgctgataaagaagatggttctccattagaatcagcagttccaccaccatttccaggtccatgtactttgggtacaggtttagctagatatgcagatttgttaaatccaccaagaaaatctgctttggttgcattagctgcatacgctactgaaccatcagaagcagaaaaattgaaacatttgacatctccagatggtaaagatgaatattcacaatggattgttgcttctcaaagatcattgttagaagttatggctgcatttccatctgctaaaccaccattgggtgttttctttgctgcaattgcaccaagattgcaaccaagatactactcaatctcttcatctccaagattggctccatctagagttcatgttacttcagcattagtttacggtccaacaccaactggtagaattcataaaggtgtttgttctacatggatgaaaaatgctgttccagcagaaaaatctcatgaatgttcaggtgctccaattttcattagagcatcaaacttcaagttgccatctaacccatcaactccaattgttatggttggtccaggtacaggtttagctccttttagaggtttcttgcaagaaagaatggcattaaaagaagatggtgaagaattgggttcatctttgttgtttttcggttgtagaaatagacaaatggatttcatctatgaagatgaattgaacaactttgttgatcaaggtgttatttctgaattgatcatggctttttcaagagaaggtgcacaaaaggaatacgttcaacataagatgatggaaaaagctgcacaagtttgggatttgattaaagaagaaggttatttgtacgtttgtggtgacgctaagggtatggcaagagatgttcatagaactttgcatacaatcgttcaagaacaagaaggtgtttcatcttcagaagctgaagcaattgttaagaaattgcaaactgaaggtagatatttgagagatgtttggtaagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttccgctacagaggagggatccccgggtacccagttgatcaaaaatcatcgcttcgctgattaattaccccagaaataaggctaaaaaactaatcgcattatcatcctatggttgttaatttgattcgttaatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtaatgccatgtaatatgattattaaacttctttgcgtccatccaaaaaaaaagtaagaatttttgaaaattcaatataaaaaaaaatggaagaaactaaatcaagattcaaaagaatctgtgttttctgtggttcttcatctggtaaaaagccatcttatcaagaagctgcaatccaattgggtaacgaattggttgaaagaagaatcgatttggtttacggtggtggttcagttggtttgatgggtttagtttctcaagctgttcatcatggtggtagacatgttttgggtgttattccaaagacattgatgccaagagaaattactggtgaaacaattggtgaagttaaagctgttgcagatatgcatcaaagaaaagctgaaatggcaagacaagctgatgcttttattgctttgccaggtggttatggtactttggaagaattgttggaagttattacatgggcacaattgggtatccatagaaagccagttggtttgttgaacgttgatggttactacaactcattgttgacttttattgataaggctgttgatgaaggttttatttcaccaatggcaagaagaatcattgtttctgctccaaacgcaaaggaattggttagacaattggaagaatacgaaccagaattcgatgaaatcacatctaagttggtttgggatgaagttgatagaatttcatacgttccaggttctgaagttgctactgcaacataagcttatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagcgctaggtaaatgaatgaggagttacgtggattgagccagcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctcctgctcaaaaaaagagggaaaaaaagctccaaaattctatcttttcctatcgctcctgtcctatccttattacgttcattactattttaatactatccattcttttattttcagtctaaaaaaaacatttctcataacgggaaaagcaaaaaaatgtcaagcttatacatcaaaacaccactgcatgcattatctgctggtccggattctcagc

SEQ ID NO: 31 - DNA, 8708 bp, synthetic construct
  source 1..8708: mol_type = other DNA
ggccgccgtctatgaggagactgttagttggatatcagtaatgagacgaaaaagctcgaaatgaatggatatattctttttgctactggcaactgttgaatatttaatgttaaaacaaactaactgaggtatattcgtatctgtatgtacacatatactatatacaggaaaagataagcaagagagaggatatcaactacgagagcgatcgattatatatcaaaagctgtccgctttgccacccataatcggcgcttagtttcggagttcaatcataattctaccaccttacactcaacttactctttaactcctatagtataatatcgccactgaccccatattaaaaaatttttttgctcgatcttctatcctctttaggttaattgtcgctgttattgtctagattttttctcggagatggcgcatctatttgccgtcaaaagatcctctcataccatattaagtaaattgcctccatttctttttcctcgggcagagaaactcgcaggcaacttgctctcgaagtggtcacgtcgctaggtatactaatgaggaggggaacaaaggattgtgcagtgccatacgacaagttcgcctcaatcgccaaaccccttgactgtgcttcccgagcggaaacatttagattggctactacaaccgttgctctccgggcggataagagaataaatctggattcggcagagtagtcggccttatcatcagttagatgttgataatcccgttgggcaggtcacacgctagcaaggagcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatcaatggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatcgctatacgctcttcatacaggaggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaaaaaaaatgatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctacagaggagccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcatcaatttctcaagtttcagtttcatttttcttgttctattacaactttttttacttcttgctcattagaaagaaagcatagcaatctaatctaagttttaattacaaaagatcaatgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctgagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttccgctaggtaaatgaatgaggagcctgcataatcggcctcacagagggatcccgttacccatctatgctgaagatttatcatactattcctccgctcgtttcttttttcagtgaggtgtgtcgtgaaagaaaacccacaattaaactttcaacaaccgggcgactaggaagagagtagtgggcgcggatgacgaaggctaaggtcacttcttcgtttcctttattggggtttccgtgtagccttcccctgaatagtgtgggacgttttatgagaagccgtaagaaataggcaaattgagttatgacaagtagacatgatgccgcagccttgcctgactttacgtctccttcatgaataagtttttctatcgagttcttttccttttttcgccttaattagctcaattaagcctgtcctcactacttttctttttcttatcggctttgtgccacacctaaccttcgaatgctgttttattccgttcttacatgggatggtaatgccttggcgaggc

SEQ ID NO: 32 - DNA, 7876 bp, synthetic construct
  source 1..7876: mol_type = other DNA
ggccgccgtctatgaggagactgttagttggatatcagtaatgagacgaaaaagctcgaaatgaatggatatattctttttgctactggcaactgttgaatatttaatgttaaaacaaactaactgaggtatattcgtatctgtatgtacacatatactatatacaggaaaagataagcaagagagaggatatcaactacgagagcgatcgattatatatcaaaagctgtccgctttgccacccataatcggcgcttagtttcggagttcaatcataattctaccaccttacactcaacttactctttaactcctatagtataatatcgccactgaccccatattaaaaaatttttttgctcgatcttctatcctctttaggttaattgtcgctgttattgtctagattttttctcggagatggcgcatctatttgccgtcaaaagatcctctcataccatattaagtaaattgcctccatttctttttcctcgggcagagaaactcgcaggcaacttgctctcgaagtggtcacgtcgctaggtatactaatgaggaggggaacaaaggattgtgcagtgccatacgacaagttcgcctcaatcgccaaaccccttgactgtgcttcccgagcggaaacatttagattggctactacaaccgttgctctccgggcggataagagaataaatctggattcggcagagtagtcggccttatcatcagttagatgttgataatcccgttgggcaggtcacacgctagcaaggagccacagagaacaggagattacaagatccagatcaccaaaatgccggaacacaggctgaaaaactccaagcagccgcacacccgatttttagccagcagattcatacctaactccaatgaattgcagcatatatcctgcaataacacattaaggatcgggccttggacggccgaatatatgattatagtccttttgtcgccaaaaagtcgcctcgaggtcacgtgcatccgaaatcaccccctgagcacttcccgcccagaaaacagggaaggggcggcaccagcaggcaccggcattcactcgtccgtgcggcgtggtctgtgccgttcaggggcctgcgtgcctgtgatgatatcgctgcagggcaccgagtcggagaggaccactccgagccgaggttagttaagccattctctacgctaaaaaataaataatgtggattaagctcctactggaaggttaagtgtttgtacactttcaaacatgcgcaacgcaccaggcaactattgttctgaggagggatgtgatgcggagtaaaatgtggaggaattacggggcttgcagacatatataaactctagccattcggctgaatataagctagaaaccacttcttctctttccatctacatctccctcatctatcaataagaactataatatcaacaatactataatataatatatacacacatttacaaaaaaaatggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagcttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatcgctatacaggagcggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaatgatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataagcttgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcgctacagaggagggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaaaaaaaaatgggccacaagaaccagaatgtcaacctccgactcagtctcgagcgggatactggactcctccgttaccagcgtgagcaggcctaccaggcccagtctcagatcctcgctcctatttcaccggtatctgtcgcgcccgtcgtctccaacggtaacggtaacgcatcgaaatcgattgagaaaccaatgcctcgtttggtggtccctaacggaccaagatccttgcctgaatcaccaccttcgacgacagaatcaaccccggtcaacaaggttatcatcggtggaccgtccgacaggcctgccctagacggactcgccaatgggaacggtgccgtcccccttgacaaacaaactgtgcttggcatgaggtcgatcgaagaatgcgaagaaattatgaagagtggactcgggccttactcactcaacgacgaagaattgattttgttgactcaaaagggaaagattccgccgtactcgctggaaaaagcattgcagaactgtgagcgggcggtcaagattcgaagggcggttatctcccgagcatccgttactaagacgctggaaacctcggacttgcccatgaaggattacgactactcgaaagtgatgggcgcatgctgtgagaacgttgtcggatatatgcctctccctgtcggaatcgctggtccacttaacattgatggcgaggtcgtccccatcccgatggccaccaccgagggaactctcgtggcctcgacgtcgagaggctgcaaagcgctcaacgcgggtggcggagtgaccaccgtcatcacccaggatgcgatgacgagaggaccggtggtggatttcccttcggtatctcaggccgcacaggccaaacgatggttggattcggtcgaaggaatggaggttatggccgcttcgttcaactcgacttctagattcgccaggttgcagagcatcaagtgtggaatggccggccgatcgctatacatccgtttggcgaccagtaccggagatgcgatgggaatgaacatggctggtaaaggaacggagaaagctttggaaaccctgtccgagtacttcccatccatgcagatccttgctctttctggtaactactgtatcgacaagaagccttctgccatcaactggattgagggccgtggaaagtccgtggtggccgagtcggtgatccctggagcgatcgtcaagtctgtcctcaagacaacggttgcggatctcgtcaacttgaacattaagaaaaacttgatcggaagtgccatggcaggcagcattggaggattcaacgcccacgcgtcgaatattttgacttcaatcttcttggctacaggtcaggatcctgcacagaatgtggagtcctcaatgtgcatgacattgatggaggctgtgaacgacggaaaagatctactcatcacctgctcgatgccggcgatcgagtgcggaacggtcggtggaggaactttcctccctccgcaaaacgcctgtttgcagatgctcggtgtcgcaggtgcccatccagattcgcccggtcacaatgctcgtcgactagcaagaatcatcgctgccagtgtgatggctggagagttgagtttgatgagtgctttggccgctggtcatttaatcaaggcccacatgaagcacaatcgatcgacaccttcgactcctctaccggtatcaccgttggcgacccgaccgaacacgccgtcccaccggtcgattggattgctcacaccgatgacgtcttccgcatcggtcgcctcgatgttctctgggttcggtagtccgtcgacgagctcgctcaagacggtaggtagcatggcttgcgtcagggaacgaggggacgagacgagtgtgaacgtggatgcctgagcttgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttccgctaggtaaatgaatgaggagcctgcataatcggcctcacagagggatcccgttacccatctatgctgaagatttatcatactattcctccgctcgtttcttttttcagtgaggtgtgtcgtgaaagaaaacccacaattaaactttcaacaaccgggcgactaggaagagagtagtgggcgcggatgacgaaggctaaggtcacttcttcgtttcctttattggggtttccgtgtagccttcccctgaatagtgtgggacgttttatgagaagccgtaagaaataggcaaattgagttatgacaagtagacatgatgccgcagccttgcctgactttacgtctccttcatgaataagtttttctatcgagttcttttccttttttcgccttaattagctcaattaagcctgtcctcactacttttctttttcttatcggctttgtgccacacctaaccttcgaatgctgttttattccgttcttacatgggatggtaatgccttggcgaggc

(19) United States
(12) Patent Application Publication
(10) Pub. No.: US 2024/0352494 A1
(43) Pub. Date: Oct. 24, 2024

(54) HETEROLOGOUS PRODUCTION OF CYTOKININS IN YEASTS

(71) Applicant: ADAMA CHILE SA, Santiago (CL)

(72) Inventors: Vincent F. CATALDO, Santiago (CL); Felipe VAREA, Santiago (CL); Andrés ARIZTÍA, Santiago (CL); Juan Manuel PUIG, Santiago (CL); Eduardo Esteban Agosin TRUMPER, Santiago (CL)

(73) Assignee: ADAMA CHILE SA, Santiago (CL)

(21) Appl. No.: 18/683,552

(22) PCT Filed: Aug. 15, 2022
(86) PCT No.: PCT/IB2022/057603

§ 371 Date: Feb. 14, 2024

Related U.S. Application Data
(60) Provisional application No. 63/233,272, filed on Aug. 15, 2021.

(51) Int. Cl. C12P 17/18; C07D 473/34; C07H 19/167; C12N 9/02; C12N 9/10; C12N 9/24

(52) U.S. Cl. CPC C12P 17/182; C07D 473/34; C07H 19/167; C12N 9/0042; C12N 9/0073; C12N 9/1085; C12N 9/2497; C12Y 106/02004; C12Y 205/01

(57) ABSTRACT

Metabolically-engineered yeast strains are provided. such as metabolically-engineered Saccharomyces cerevisiaestrains. producing high amounts of at least one. preferably all four natural cytokinins: trans-zeatin (tZ), trans-zeatin riboside (tZR). isopentenyladenine (iP) and isopenteny ladenine riboside (iPR).

### FIELD OF THE INVENTION

The present invention relates to metabolic-engineered yeast strains, such as metabolic-engineered Saccharomyces cerevisiae strains, producing high amounts of at least one, preferably all four natural cytokinins: trans-zeatin (tZ), trans-zeatin riboside (tZR), isopentenyladenine (iP) and isopentenyladenine riboside (iPR).

### BACKGROUND OF THE INVENTION

Cytokinins are a family of phytohormones that regulate plant growth processes. Cytokinins have a wide range of applications in crop improvement and management, such as micropropagation, general plant growth and health, and regulation of fruit size and quality (Aremu et al., 2020, Biomolecules, 10(9); Koprna et al., 2016, Bioorganic Med. Chem., 24:484-492). Trans-zeatin (tZ) and isopentenyladenine (iP) together with their sugar conjugates (glucosides and ribosides) are the most prevalent natural cytokinins. The free-base forms (tZ and iP) have stronger biological activities and higher receptor affinities than their sugar conjugates (Hoyerová and Hošek, 2020, Plant Sci., 11: 5-11; Kieber and Schaller, 2014, Arab. B., 12: c0168). However, cytokinin nucleosides such as trans-zeatin riboside (tZR) and isopentenyladenine riboside (iPR), have shown capacity to activate some specific receptors. Thus, these cytokinin nucleosides are considered as a translocation form of cytokinins between plant tissues, and can be converted into free-base forms (Sakakibara, 2006, Annu. Rev. Plant Biol., 57:431-449). Despite their potential benefits, the current high price of commercial cytokinins hampers wide applications in agriculture.

Some microorganisms, including various species of yeasts, naturally produce cytokinins. For example, Streletskii et al., (2019, PeerJ., 7: c6474), tested a collection of natural yeast strains and detected Z production in more than a half (55%) of studied strains. However, the production yields are very low (ng/g of biomass) and/or the microorganisms are not suitable for large-scale cultivation for commercial purposes.

Cytokinins precursors are obtained through the mevalonate pathway, which synthesizes isoprenoids used in a wide range of physiological processes in eukaryotes, archaea and some bacteria. A key enzyme in the isoprenoid and sterol synthesis is mevalonate kinase, which has been reported to participate in cytokinin-mediated growth and development as well as in substrate feedback and diurnal regulation (Miziorko, 2011, Archives of Biochemistry and Biophysics 505:131-143; Kasahara et al., 2004, Journal of Biological Chemistry 279:14049-14054; Åstot et al., 2000, PANS USA 97:14778-14783).

Takei et al. (2014, J. Biol. Chem., 279 (40): 41866-72) studied the biosynthesis of trans-zeatin and demonstrated that the cytokinin hydroxylases CYP735A1 and CYP735A2 are cytochrome P450 monooxygenases (P450s) that catalyze the biosynthesis of tZ. Takei et al. identified the genes from Arabidopsis using an adenosine phosphate isopentenyltransferase (AtIPT4)/P450 co-expression system in yeast. These strains were able to produce up to 3.36 mg/L of total cytokinins (tZ+(ZR+iP+iPR) in shake flask cultures.

European Patent Application Publication No. EP0248984 discloses the production of cytokinins, particularly tZ, by bacterial cells containing plasmids with insert DNA (tmr and/or tzs) isolated from Ti plasmids of Agrobacterium tumefaciens or with insert DNA isolated from a plasmid of Pseudomonas syringae pv. savastanoi (P. savastanoi). When both tmr and tza are included in the same plasmid, there is a substantial elevation in the tZ yield. This approach yielded a maximum concentration of 0.11 mg/L of tZ and 0.015 mg/L of iP.

These data reveal the necessity to improve the biosynthesis of cytokinins in microorganisms at high concentrations and productivities, enough to meet industrial standards and positioning as a feasible production method.

### SUMMARY OF THE INVENTION

The present invention is directed to methodologies to produce natural cytokinins by assembly of plant-derived cytokinin pathway in yeast cells using synthetic biology and metabolic engineering tools. As exemplified hereinbelow, the genomic integration of four heterologous biosynthetic genes under galactose-inducible promoters enabled the production of large amounts of four cytokinins in yeast cells: trans-zeatin (tZ), trans-zeatin riboside (tZR), isopentenyladenine (iP), and isopentenyladenine riboside (iPR). Enhancement of precursor supply through overexpression of mevalonate pathway genes further increased cytokinin biosynthesis.

The present invention also discloses a fed-batch fermentation bioprocess of cytokinin-producing strains. The robust production method disclosed herein is based on a glucose growth phase followed by a galactose induction phase with intermittent ethanol pulses. This strategy yielded 1.5 g/L of total cytokinins with a volumetric productivity of 20 mg/L/h.

According to certain aspects, the present invention provides a genetically-modified yeast producing at least cytokinin selected from the group consisting of trans-zeatin, trans-zeatin riboside, isopentenyladenine tzar riboside and any combination thereof, the genetically modified yeast comprises a plurality of exogenous polynucleotides comprising:
(i) a polynucleotide encoding an isopentenyl transferase;
(ii) a polynucleotide encoding a cytokinin hydroxylase;
(iii) a polynucleotide encoding an NADPH-cytochrome P450 reductase; and
(iv) a polynucleotide encoding a cytokinin riboside 5′-monophosphate phosphoribohydrolase.

According to certain embodiments, the encoded enzymes comprise amino acid sequence of plant enzymes, fungal enzymes, homologs thereof or any combination thereof. Each possibility represents a separate embodiment of the present invention.

According to certain embodiments, the encoded enzymes are of a plant origin or enzymes homologous thereto.

According to certain exemplary embodiments, the plant is Arabidopsis thaliana.

In some embodiments, the encoded isopentenyl transferase comprises an amino acid sequence having at least 80% identity to the amino acid sequence of A. thaliana IPT4. In some embodiments, the encoded isopentenyl transferase comprises the amino acid sequence of A. thaliana IPT4. In some embodiments, the A. thaliana IPT4 comprises the amino acid sequence set forth in SEQ ID NO:1.

In some embodiments, the encoded cytokinin hydroxylase comprises an amino acid sequence having at least 80% identity to the amino acid sequence of A. thaliana CYP735A1. In some embodiments, the encoded cytokinin hydroxylase comprises the amino acid sequence of A. thaliana CYP735A1. In some embodiments, the A. thaliana CYP735A1 comprises the amino acid sequence set forth in SEQ ID NO:2.

In some embodiments, the NADPH-cytochrome P450 reductase comprises an amino acid sequence having at least 80% identity to the amino acid sequence of A. thaliana ATR1. In some embodiments, the encoded NADPH-cytochrome P450 reductase comprises the amino acid sequence of the A. thaliana ATR1. In some embodiments, the A. thaliana ATR1 comprises the amino acid sequence set forth in SEQ ID NO:3.

In some embodiments, the cytokinin riboside 5′-monophosphate phosphoribohydrolase comprises an amino acid sequence having at least 80% identity to the amino acid sequence of A. thaliana LOG7. In some embodiments, the encoded cytokinin riboside 5′-monophosphate phosphoribohydrolase comprises the amino acid sequence of A. thaliana LOG7. In some embodiments, the A. thaliana LOG7 comprises the amino acid sequence set forth in SEQ ID NO:4.

In some embodiments, the genetically-modified yeast comprises: (i) an exogenous polynucleotide encoding an isopentenyl transferase having at least 80% identity to the amino acid sequence of A. thaliana IPT4; (ii) an exogenous polynucleotide encoding a cytokinin hydroxylase having at least 80% identity to the amino acid sequence of A. thalianaCYP735A1; (iii) an exogenous polynucleotide encoding NADPH-cytochrome P450 reductase having at least 80% identity to the amino acid sequence of A. thaliana ATR1; and
(iv) an exogenous polynucleotide encoding a cytokinin riboside 5′-monophosphate phosphoribohydrolase having at least 80% identity to the amino acid sequence of A. thalianaLOG7.

In some embodiments, the genetically-modified yeast comprises: (i) an exogenous polynucleotide encoding A. thaliana IPT4; (ii) an exogenous polynucleotide encoding A. thaliana CYP735A1; (iii) an exogenous polynucleotide encoding A. thaliana ATR1; and (iv) an exogenous polynucleotide encoding A. thaliana LOG7.

In some embodiments, the yeast is Saccharomyces cerevisiae and the polynucleotides are optimized for expression in this yeast. In some embodiments, the yeast is Saccharomyces cerevisiae comprising an exogenous polynucleotide encoding A. thalianaIPT4 comprising the nucleic acid sequence set forth in SEQ ID NO:8. In some embodiments, the yeast is Saccharomyces cerevisiae comprising an exogenous polynucleotide encoding

A. thaliana CYP735A1 comprising the nucleic acid sequence set forth in SEQ ID NO:9. In some embodiments, the yeast is Saccharomyces cerevisiae comprising an exogenous polynucleotide encoding A. thaliana ATR1 comprising the nucleic acid sequence set forth in SEQ ID NO:10. In some embodiments, the yeast is Saccharomyces cerevisiae comprising an exogenous polynucleotide encoding A. thaliana LOG7 comprising the nucleic acid sequence set forth in SEQ ID NO: 11.

In some embodiments, the yeast is Saccharomyces cerevisiae comprising: an heterologous polynucleotide encoding A. thaliana IPT4 comprising the nucleic acid sequence set forth in SEQ ID NO:8; an heterologous polynucleotide encoding A. thaliana CYP735A1 comprising the nucleic acid sequence set forth in SEQ ID NO:9; an heterologous polynucleotide encoding A. thaliana ATR1 comprising the nucleic acid sequence set forth in SEQ ID NO:10; and a heterologous polynucleotide encoding A. thaliana LOG7 comprising the nucleic acid sequence set forth in SEQ ID NO:11.

In some embodiments, each of the plurality of the exogenous polynucleotides or a combination thereof is comprised within an expression cassette further comprising at least one regulatory element. The at least one regulatory element can be operably linked to each of the exogenous polynucleotides a combination of the exogenous polynucleotides can be operably linked to a single at least one regulatory element. The combination may include two, three or all four heterologous polynucleotides.

In some embodiments, the regulatory element is selected from a promoter, an enhancer, a termination sequence and any combination thereof.

In some embodiments, the promoter is an inducible promoter. In some particular embodiments, the promoter is a galactose-inducible promoter.

In some embodiments, the yeast is Saccharomyces cerevisiae comprising: an expression cassette encoding A. thaliana IPT4 comprising the nucleic acid sequence set forth in SEQ ID NO: 15; an expression cassette encoding A. thaliana CYP735A1 comprising the nucleic acid sequence set forth in SEQ ID NO: 16; an expression cassette encoding A. thaliana ATR1 comprising the nucleic acid sequence set forth in SEQ ID NO:17; and an expression cassette encoding A. thaliana LOG7 comprising the nucleic acid sequence set forth in SEQ ID NO:18. In some embodiments, the expression cassettes are arranged in a single polynucleotide molecule. In some particular embodiments, the single polynucleotide molecule comprises the nucleic acid sequence set forth in SEQ ID NO:25.

In some embodiments, a genetically-modified yeast of the present invention further comprises at least one additional exogenous polynucleotide selected from the group consisting of;
(A) one or more exogenous polynucleotides encoding an amino-terminal truncated HMG-COA reductase lacking the transmembrane domain; and
(B) an exogenous polynucleotide encoding an isopentenyl isomerase.

In some embodiments, the genetically-modified yeast further comprises: an exogenous polynucleotide encoding an amino-terminal truncated HMG-COA reductase having at least 80% identity to the amino acid sequence of Saccharomyces cerevisiae amino-terminal truncated HMG-COA reductase (Sc-tHMG1); and an exogenous polynucleotide encoding an amino-terminal truncated HMG-COA reductase 1 having at least 80% identity to the amino acid sequence of Xanthophyllomyces dendrorhous amino-terminal truncated HMG-COA reductase (Xd-tHMG1).

In some embodiments, the genetically-modified yeast further comprises an exogenous polynucleotide encoding Sc-tHMG1 and an exogenous polynucleotide encoding Xd-tHMG1.

In some exemplary embodiments, the Sc-tHMG1 comprises the amino acid sequence set forth in SEQ ID NO:5, and the Xd-tHMG1 comprises the amino acid sequence set forth in SEQ ID NO:6.

In some embodiments, the isopentenyl isomerase comprises an amino acid sequence having at least 80% identity to the amino acid sequence of S. cerevisiae isopentenyl isomerase (IDI1). In some embodiments, the isopentenyl isomerase is S. cerevisiae IDI1. In some particular embodiments, the S. cerevisiae IDI1 comprises the amino acid sequence set forth in SEQ ID NO:7.

In some embodiments, the genetically-modified yeast further comprises:
(A) an exogenous polynucleotide encoding a truncated HMG-COA reductase having at least 80% identity to the amino acid sequence of Sc-tHMG1;
(B) an exogenous polynucleotide encoding a truncated HMG-COA reductase having at least 80% identity to the amino acid sequence of Xd-tHMG1; and
(C) an exogenous polynucleotide encoding an isopentenyl isomerase having at least 80% identity to the amino acid sequence of S. cerevisiae IDI1.

In some embodiments, the genetically-modified yeast further comprises:
(A) an exogenous polynucleotide encoding Sc-tHMG1;
(B) an exogenous polynucleotide encoding Xd-tHMG1; and
(C) an exogenous polynucleotide encoding S. cerevisiae IDI1.

In some embodiments, the genetically-modified yeast comprises: (i) an exogenous polynucleotide encoding A. thaliana IPT4; (ii) an exogenous polynucleotide encoding A. thaliana CYP735A1; (iii) an exogenous polynucleotide encoding A. thaliana ATR1; and (iv) an exogenous polynucleotide encoding A. thaliana LOG7; (v) an exogenous polynucleotide encoding Sc-tHMG1; (vi) an exogenous polynucleotide encoding Xd-tHMG1; and (vii) an exogenous polynucleotide encoding S. cerevisiae IDI1

In some embodiments, the yeast is Saccharomyces cerevisiae and the polynucleotides are codon optimized for expression in this yeast. In some embodiments, the yeast is Saccharomyces cerevisiae comprising an exogenous polynucleotide encoding Sc-tHMG1 comprising the nucleic acid sequence set forth in SEQ ID NO:12. In some embodiments, the yeast is Saccharomyces cerevisiae comprising an exogenous polynucleotide encoding Xd-tHMG1 comprising the nucleic acid sequence set forth in SEQ ID NO: 13. In some embodiments, the yeast is Saccharomyces cerevisiae comprising an exogenous polynucleotide encoding S. cerevisiae IDI1 comprising the nucleic acid sequence set forth in SEQ ID NO:14.

In some embodiments, the yeast is Saccharomyces cerevisiae further comprising: an exogenous polynucleotide encoding Sc-tHMG1 comprising the nucleic acid sequence set forth in SEQ ID NO:12; an exogenous polynucleotide encoding Xd-tHMG1 comprising the nucleic acid sequence set forth in SEQ ID NO:13; and an exogenous polynucleotide encoding S. cerevisiae IDI1 comprising the nucleic acid sequence set forth in SEQ ID NO: 14.

In some embodiments, each of the plurality of the exogenous polynucleotides or a combination thereof is comprised within an expression cassette further comprising at least one regulatory element. In some embodiments, the regulatory element is selected from a promoter, an enhancer, a termination sequence and any combination thereof. In some embodiments, the promoter is an inducible promoter. In some particular embodiments, the promoter is a galactose-inducible promoter. In other particular embodiments, the promoter is a constitutive promoter.

In some embodiments, the yeast is Saccharomyces cerevisiae further comprising: an expression cassette encoding Sc-tHMG1 comprising the nucleic acid sequence set forth in SEQ ID NO:22; an expression cassette encoding Xd-tHMG1 comprising the nucleic acid sequence set forth in SEQ ID NO:23; and an expression cassette encoding S. cerevisiae IDI1 comprising the nucleic acid sequence set forth in SEQ ID NO:24.

In some embodiments, the yeast is Saccharomyces cerevisiae further comprising: an expression cassette encoding Sc-tHMG1 comprising the nucleic acid sequence set forth in SEQ ID NO: 19; an expression cassette encoding Xd-tHMG1 comprising the nucleic acid sequence set forth in SEQ ID NO: 20; and an expression cassette encoding S. cerevisiaeIDI1 comprising the nucleic acid sequence set forth in SEQ ID NO: 21.

In some embodiments, the expression cassettes are arranged in a single polynucleotide molecule. In some particular embodiments, the single polynucleotide molecule comprises a nucleic acid sequence selected from SEQ ID NO: 27 and SEQ ID NO: 28. Each possibility represents a separate embodiment of the present invention.

In some embodiments, a genetically-modified yeast of the present invention comprises at least one additional copy of one or more of the exogenous polynucleotides encoding the cytokinin hydroxylase, NADPH-cytochrome P450 reductase and cytokinin riboside 5′-monophosphate phosphoribohydrolase.

In some embodiments, a genetically-modified yeast of the present invention comprises an additional copy of each of the exogenous polynucleotides encoding the cytokinin hydroxylase, NADPH-cytochrome P450 reductase and cytokinin riboside 5′-monophosphate phosphoribohydrolase.

In some embodiments, the additional copies of the heterologous polynucleotides encoding the cytokinin hydroxylase, NADPH-cytochrome P450 reductase and cytokinin riboside 5′-monophosphate phosphoribohydrolase are arranged in a single polynucleotide molecule. In some embodiments, the single polynucleotide molecule comprising the nucleic acid sequence set forth in SEQ ID NO:26.

In some embodiments, the yeast is Saccharomyces cerevisiae further comprising: an exogenous polynucleotide comprising the sequence set forth in SEQ ID NO:25 and an exogenous polynucleotide comprising the sequence set forth in SEQ ID NO:28. In some embodiments, the yeast further comprises an exogenous polynucleotide comprising the sequence set forth in SEQ ID NO:26.

According to another aspect, the present invention provides a method for producing at least one cytokinin selected from the group consisting of trans-zeatin, trans-zeatin riboside, isopentenyladenine and isopentenyladenine riboside and any combination thereof, the method comprising:
(A) providing a yeast genetically-modified according to the present invention;
(B) culturing the genetically-modified yeast in a culture medium under conditions suitable for expression of the exogenous polynucleotides; and
(C) recovering the one or more cytokinins from the medium.

In some embodiments, step (A) comprises providing a genetically-modified yeast in which the exogenous polynucleotides are expressed under a galactose-inducible promoter, and the culturing in step (B) is performed by fed-batch fermentation process comprising a glucose growth phase followed by a galactose induction phase with intermittent ethanol pulses.

In some embodiments, at least 60% (w/w) of the produced cytokinins are trans-zeatin and isopentenyladenine. In additional embodiments, at least 70% (w/w) of the produced cytokinins are trans-zeatin and isopentenyladenine.

According to a further aspect, the present invention provides an expression cassette for genetically-modifying a yeast, comprising at least one regulatory element operably-linked to a nucleic acid sequence selected from the group consisting of:
(b) a nucleic acid sequence encoding a cytokinin hydroxylase;
(c) a nucleic acid sequence encoding an NADPH-cytochrome P450 reductase; and
(d) a nucleic acid sequence encoding (a) a nucleic acid sequence encoding an isopentenyl transferase;
a cytokinin riboside 5′-monophosphate phosphoribohydrolase.

According to certain embodiments, the encoded enzymes comprise amino acid sequence of plant enzymes, fungal enzymes, a combination thereof and homologs thereto. Each possibility represents a separate embodiment of the present invention.

According to certain embodiments, the encoded enzymes are of a plant origin or enzymes homologous thereto.

According to certain embodiments, the plant is A. thaliana.

According to certain embodiments, the nucleic acid sequence encoding the A. thaliana enzyme is codon optimized for expression in Saccharomyces cerevisiae.

In some exemplary embodiments, the nucleic acid sequence is selected from the group consisting of:
(a) a nucleic acid sequence encoding A. thaliana IPT4 comprising the sequence set forth in SEQ ID NO:8;
(b) a nucleic acid sequence encoding A. thaliana CYP735A1 comprising the sequence set forth in SEQ ID NO:9;
(c) a nucleic acid sequence encoding A. thaliana ATR1 comprising the sequence set forth in SEQ ID NO:10; and
(d) a nucleic acid sequence encoding A. thaliana LOG7 comprising the sequence set forth in SEQ ID NO:11.

In some embodiments, the at least one regulatory element comprises an inducible promoter. In some embodiments, the promoter is a galactose-inducible promoter. In some embodiments, the promoter is a galactose-inducible promoter and the expression cassette is selected from the group consisting of: an expression cassette for expressing A. thaliana IPT4 comprising the sequence set forth in SEQ ID NO:15; an expression cassette for expressing A. thaliana CYP735A1 comprising the sequence set forth in SEQ ID NO:16; an expression cassette for expressing A. thaliana ATR1 comprising the sequence set forth in SEQ ID NO: 17; and an expression cassette for expressing A. thaliana LOG7 comprising the sequence set forth in SEQ ID NO:18.

According to a further aspect, the present invention provides a nucleic acid construct for genetically-modifying a yeast to produce cytokinins comprising a plurality of expression cassettes, the plurality of expression cassettes comprising: an expression cassette for expressing A. thaliana IPT4 comprising the sequence set forth in SEQ ID NO: 15; an expression cassette for expressing A. thaliana CYP735A1 comprising the sequence set forth in SEQ ID NO:16; an expression cassette for expressing A. thaliana ATR1 comprising the sequence set forth in SEQ ID NO: 17; and an expression cassette for expressing A. thaliana LOG7 comprising the sequence set forth in SEQ ID NO:18.

In some embodiments, there is provided herein a vector for transforming a yeast cell to produce cytokinins, the vector comprising the nucleic acid construct, flanked by 5′ and 3′ yeast genomic integrating sequences. In some particular embodiments, the vector comprises a sequence selected from SEQ ID NO:29 and SEQ ID NO:30. Each possibility represents a separate embodiment of the present invention.

According a further aspect, the present invention provides a yeast that produces one or more of the following cytokinins: trans-zeatin, trans-zeatin riboside, isopentenyladenine and isopentenyladenine riboside, the method comprising transforming the yeast with a plurality of exogenous polynucleotides according to the present invention.

In some embodiments, the method further comprises transforming the yeast with at least one additional exogenous polynucleotide selected from the group consisting of: (A) one or more exogenous polynucleotide encoding an amino-terminal truncated HMG-COA reductase lacking the transmembrane domain; and (B) an exogenous polynucleotide encoding an isopentenyl isomerase.

In some embodiments, the yeast is further transformed with:
an exogenous polynucleotide encoding an amino-terminal truncated HMG-COA reductase 1 of Saccharomyces cerevisiae (Sc-tHMG1) or a truncated HMG-COA reductase having at least 80% identity to the amino acid sequence of Sc-tHMG1; and/or an exogenous polynucleotide encoding an amino-terminal truncated HMG-COA reductase 1 of Xanthophyllomyces dendrorhous (Xd-tHMG1) or a truncated HMG-COA reductase having at least 80% identity to the amino acid sequence of Xd-tHMG1; and an exogenous polynucleotide encoding isopentenyl isomerase IDI1 of S. cerevisiaeor an isopentenyl isomerase having at least 80% identity to the amino acid sequence of S. cerevisiae IDI1.

In some particular embodiments, the yeast is transformed with: an exogenous polynucleotide encoding Sc-tHMG1 comprising the nucleic acid sequence set forth in SEQ ID NO: 12; an exogenous polynucleotide encoding Xd-tHMG1 comprising the nucleic acid sequence set forth in SEQ ID NO:13; and an exogenous polynucleotide encoding S. cerevisiae IDI1 comprising the nucleic acid sequence set forth in SEQ ID NO: 14.

In some embodiments, the method further comprises transforming the yeast with an additional copy of each of the exogenous polynucleotides encoding the cytokinin hydroxylase, NADPH-cytochrome P450 reductase and cytokinin riboside 5′-monophosphate phosphoribohydrolase.

In some embodiments, the yeast is Saccharomyces cerevisiae.

These and further aspects and features of the present invention will become apparent from the detailed description, examples, and claims which follow.

### BRIEF DESCRIPTION OF THE FIGURES

FIG. 1. Shows the chemical structures of the four cytokinins produced by genetically-modified yeasts of the present invention: trans-zeatin (tZ), trans-zeatin riboside (tZR), Isopentenyladenine (iP), Isopentenyladenine riboside (iPR).

FIG. 2 demonstrates the heterologous cytokinin pathway assembled in S. cerevisiaeaccording to certain embodiments of the invention. The cytokinin pathway was constructed by expressing at least four Arabidopsis thaliana genes: IPT4, CYP735A1, ATR1, and LOG7. Solid arrows represent native reactions catalyzed by endogenous S. cerevisiaeenzymes, while dashed arrows show the heterologous reactions catalyzed by the introduced plant enzymes.

FIG. 3 shows an example of Level 3 vector (CK3.1), which joins up four transcriptional units required for cytokinin biosynthetic pathway assembly. The transcriptional units are flanked by genomic integration sites, enabling the insertion of the construct into specific chromosomal sites of the yeast using CRISPR/Cas9.

FIG. 4 shows HPLC-DAD analysis of four cytokinins in fermented yeast culture media obtained from shake flask cultures of CK1 strain. Top chromatogram: a standard mixture of trans-zeatin (tZ), trans-zeatin riboside (tZR), isopentenyladenine riboside (iPR) and isopentenyladenine (iP). Second chromatogram: fermented medium from the culture of CK1 in the absence of induction (YPD medium). Third chromatogram: fermented medium from the culture of CK1 in the presence of induction (YPDG medium). Bottom chromatogram: YPDG medium spiked with the standard mixture.

FIG. 5 shows cytokinin production in shake flask cultures of CK1-A strain using different induction culture media. The strain was cultivated for 48 h at 30° C. The carbon source was constant at 2% sugar in the medium but with different glucose and galactose proportions. YPG (2%): 2% galactose. YDPG (1.5%): 0.5% glucose, 1.5% galactose. YPDG (1%): 1% glucose, 1% galactose. tZ: trans-zeatin. tZR: trans-zeatin riboside. iP: isopentenyladenine. iPR: isopentenyladenine riboside

FIG. 6 shows cytokinin production in shake flask cultures of CK1-B strain using different induction culture media. The strain was cultivated for 48 h at 30° C. The carbon source was constant at 2% sugar in the medium but with different glucose and galactose proportions. YPG (2%): 2% galactose. YDPG (1.5%): 0.5% glucose, 1.5% galactose. YPDG (1%): 1% glucose, 1% galactose. tZ: trans-zeatin. tZR: trans-zeatin riboside. iP: isopentenyladenine. iPR: isopentenyladenine riboside

FIG. 7 shows cytokinin production in 1 L batch bioreactor fermentation of strain CK1-B. The culture started with 1.5% of glucose and 1.5% of galactose. A galactose pulse (20 g/L) was added after 32 h of fermentation. Total cytokinin concentration is the sum of trans-zeatin, trans-zeatin riboside, isopentenyladenine, and isopentenyladenine riboside.

FIG. 8 shows cytokinin production in CK2.1, CK2.2 and CK3.1 strains. The strains were cultivated in shake flask containing YPDG media (1% glucose, 1% galactose) for 48 h at 30° C.

FIG. 9 shows cytokinin production by fed-batch fermentation of CK2.2 strain. The strategy consisted of a glucose-limited growth phase followed by a galactose induction phase. An adaptation to galactose was carried out before the change of carbon sources by the administration of a galactose pulse (15 g/L) at 22 h of the growth phase (triangle-headed arrow). At 30 h of fermentation, the growth medium feeding was replaced by the induction medium (round-headed arrow). Periodic pulses of ethanol (15 g/L) were administered every 10 h (square-headed arrow) to promotes Acetyl-CoA supply and cytokinin production.

FIG. 10 shows detailed cytokinin composition throughout fed-batch fermentation of CK2.2 strain. tZ: trans-zeatin. tZR: trans-zeatin riboside. iP: isopentenyladenine. iPR: isopentenyladenine riboside. Total CK: total cytokinins.

FIG. 11 demonstrates the biological activity of the cytokinins produced in yeast. The activity was measured by inhibition of hypocotyl elongation in A. thaliana. FIG. 11A shows a representative picture of the hypocotyl and FIG. 11B shows a quantitative analysis of the hypocotyl length. tZ: trans-zeatin. Blank: 0 mg/L of tZ. CK2.2: culture medium obtained by fed-batch fermentation of CK2.2 strain, diluted 1/10000 to reach 0.12 mg/L of total cytokinins. C−: negative control consisting of fermented culture medium of the parent strain CEN.PK113-5D.

### DETAILED DESCRIPTION OF THE INVENTION

The present invention provides metabolically-engineered yeast strains, such as metabolically-engineered Saccharomyces cerevisiae strains, producing high amounts of at least one natural cytokinins, preferably four natural cytokinins selected from the group consisting of:
trans-zeatin (tZ), CAS: 1637-39-4
trans-zeatin riboside (tZR), CAS: 6025-53-2
Isopentenyladenine (IP), CAS: 2365-40-4
Isopentenyladenine riboside (iPR): CAS: 7724-76-7

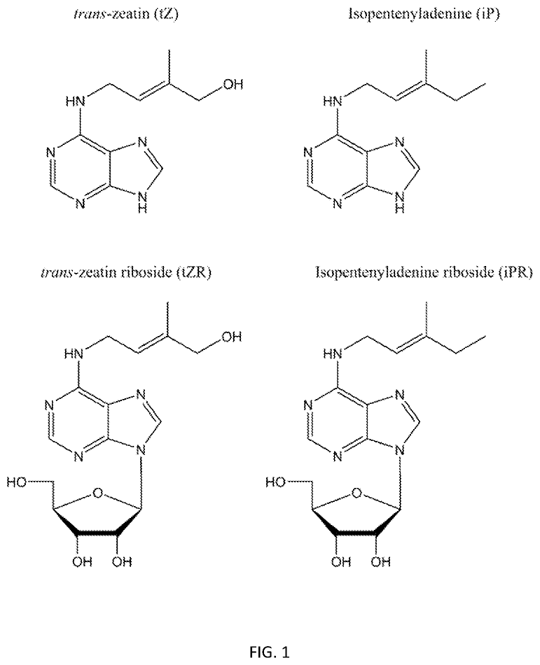

Trans-zeatin ((Z), trans-zeatin riboside (tZR), isopentenyladenine (iP) and isopentenyladenine riboside (iPR) belongs to the isoprenoids cytokinin family. These cytokinins are prenylated derivatives of adenine (FIG. 1). They can be mainly present in free-base form (tZ and iP) or attached to a ribose sugar as nucleosides forms (tZR and iPR).

In plants, cytokinins are biosynthesized starting from ATP or ADP. First, adenosine phosphate-isopentenyltransferases (IPT), catalyzes the N-prenylation of ADP or ATP using dimethylallyl diphosphate (DMAPP) as prenyl donor to form isopentenyl nucleotides (iPRTP, iPRDP). These isopentenyl nucleotides can be hydroxylated at the terminal carbon of prenyl side chain by specific cytokinin hydroxylase (cytochrome P450 monooxygenases family), generating trans-zeatin nucleotides (tZRTP, tZRDP, and (ZRMP). The tri-and di-phosphorylated iP and tZ nucleotides are dephosphorylated to iPRMP and tZRMP by enzymes of nucleotide metabolism. These monophosphate CK-nucleotides can be dephosphorylated again to yield the cytokinins nucleosides forms (tZR and iPR), or LOG enzymes can directly hydrolyze the phospho-ribose group to produce the free cytokinins (tZ and iP) (Kamada-Nobusada and Sakakibara, 2009, Phytochemistry, 70: 444-449; Kieber and Schaller, 2014, Arab. B., 12, c0168).

Several fungi species, particularly plant-pathogenic fungi, are also capable to produce cytokinins (e.g., Eisermann et al., 2020. Fungal Genetics and Biology 143:103436; Chanclud et al., 2016. PLOS Pathogens 12(2): e1005457).

The construction of cytokinin-producing yeast strains as disclosed herein comprises the expression of at least four heterologous, genes of plant origin: an isopentenyl transferase (EC 2.5.1.112), a cytokinin hydroxylase (EC 1.14.13), an NADPH-cytochrome P450 reductase (EC 1.6.2.4), and a cytokinin riboside 5′-monophosphate phosphoribohydrolase (EC 3.2.2.n1). The expression of an NADPH-cytochrome P450 reductase is intended to promote a more efficient electron transfer between NADPH and the cytokinin hydroxylase.

In some embodiments, the genes are Arabidopsis thaliana genes or polynucleotides homologous thereto. In certain embodiments, yeasts of the present invention comprise exogenous polynucleotides encoding enzymes having at least 80% identity to the amino acid sequence of A. thaliana enzymes, for example at least 85%, at least 90%, at least 95%, at least 97%, at least 99% or 100% identity to the amino acid sequence of A. thaliana enzymes. Each possibility represents a separate embodiment of the present invention.

In some embodiments, yeasts of the present invention comprise exogenous polynucleotides encoding A. thaliana enzymes.

In some particular embodiments, cytokinin-producing yeasts of the present invention comprise exogenous polynucleotides encoding the following A. thaliana enzymes: isopentenyl transferase 4 (IPT4), cytokinin hydroxylase CYP735A1, NADPH-cytochrome P450 reductase ATR1, and cytokinin riboside 5′-monophosphate phosphoribohydrolase 7 (LOG7).

As disclosed herein, product yield and productivities of cytokinin-producing yeasts of the present invention are improved by increasing DMAPP supply through MVA pathway enhancement. In some embodiments, cytokinin-producing yeasts of the present invention are further genetically modified by overexpression of amino-terminal truncated version of HMG-COA reductase (tHMG1) (EC 1.1.1.34) and isopentenyl isomerase (IDI1) (EC 5.3.3.2). In additional embodiments, cytokinin-producing yeasts of the present invention are further genetically modified by expression of additional copies of cytokinin biosynthetic genes. As disclosed herein, the latter is useful for controlling cytokinin proportions.

To further increase cytokinin concentrations and volumetric productivities, the cytokinin-producing yeasts of the present invention may be grown in a fed-batch fermentation process comprising a glucose growth phase followed by a galactose induction phase with intermittent ethanol pulses. A glucose exponential growth phase comprises culturing the cells in a medium containing glucose as a carbon source until high biomass is reached. A galactose induction phase with intermittent ethanol pulses comprises culturing the cells in a medium containing galactose as a carbon source, and providing ethanol to the medium at predefined intervals. In some embodiments, the intervals are defined based on an approximate consumption rate by the cell culture. In some embodiments, the intervals are between 5-15 hours, including each value within the range, for example between 10-12 hours, including each value within the range. In some exemplary embodiments, intervals of 10 hours are selected.

In some embodiments, at least 60% (w/w) of the total cytokinins produced according to the present invention are trans-zeatin and isopentenyladenine. In additional embodiments, 60-80% of the total cytokinins produced according to the present invention are trans-zeatin and isopentenyladenine. In additional embodiments, at least 70% (w/w) of the total cytokinins produced according to the present invention are trans-zeatin and isopentenyladenine, for example, at least 75%, at least 78%, between 70%-85%, between 75%-85%, between 70%-80% or between 75%-80% trans-zeatin and isopentenyladenine out of the total cytokinins (w/w). Each possibility represents a separate embodiment of the present invention.

Yeast species for use according to the present invention include S. cerevisiae, Pichia pastoris, Hansenula polymorpha, Kluyveromyces lactis and Yarrowia lipolytica. Each possibility represents a separate embodiment of the present invention. In some embodiments, a yeast of the present invention is S. cerevisiae.

In some embodiments, the genetically-modified yeasts of the present invention produce the cytokinins at increased amounts compared to a corresponding non-genetically modified yeast.

The term “heterologous”, when referring to a gene or a protein (polynucleotide or polypeptide), is used herein to describe a gene/polynucleotide or a protein/polypeptide that is not naturally found or expressed in the specific organism being referred to as expressing the polynucleotide or polypeptide, for example a yeast transformed according to the present invention.

The term “exogenous”, when referring to a polynucleotide, is used herein to describe a synthetic polynucleotide that is exogenously introduced into a yeast cell via transformation, so as to produce a ribonucleic acid (RNA) molecule and subsequently a polypeptide molecule. The exogenous polynucleotide can be heterologous polynucleotide as defined hereinabove or an endogenous polynucleotide exogenously introduced into the yeast cell, either under endogenous or exogenous regulatory elements. The exogenous polynucleotide may be introduced into the yeast in a stable or transient manner, so as to produce a ribonucleic acid (RNA) molecule and/or a polypeptide molecule. The term “endogenous” as used herein refers to a polynucleotide or polypeptide which is naturally present and/or naturally expressed within a specific organism.

The term “expression cassette” is used herein to describe an artificially assembled nucleic acid molecule which includes a nucleic acid sequence encoding a protein of interest and which is assembled such that the protein of interest is expressed in a target host cell. An expression cassette typically comprises appropriate regulatory sequences operably linked to the nucleic acid sequence encoding the protein of interest. An expression cassette may further include a nucleic acid sequence encoding a selection marker.

The terms “nucleic acid sequence”, “nucleotide sequence” and “polynucleotide” are used herein to refer to polymers of deoxyribonucleotides (DNA), ribonucleotides (RNA), and modified forms thereof in the form of a separate fragment or as a component of a larger construct. A nucleic acid sequence may be a coding sequence, i.e., a sequence that encodes for an end product in the cell, such as a protein. A nucleic acid sequence may also be a regulatory sequence, such as, for example, a promoter.

The terms “peptide”, “polypeptide” and “protein” are used herein to refer to a polymer of amino acid residues. The term “peptide” typically indicates an amino acid sequence consisting of 2 to 50 amino acids, while “protein” indicates an amino acid sequence consisting of more than 50 amino acid residues.

A sequence (such as a nucleic acid sequence and an amino acid sequence) that is “homologous” to a reference sequence refers herein to percent identity between the sequences, where the percent identity is at least 80%, at least 85%, at least 90%, at least 95%, at least 98% or at least 99%. Each possibility represents a separate embodiment of the present invention. Homologs of the sequences described herein are encompassed within the present invention. Protein homologs are encompassed as long as they maintain the activity of the original protein. Homologous nucleic acid sequences include variations related to codon usage and degeneration of the genetic code. Sequence identity may be determined using nucleotide/amino acid sequence comparison algorithms, as known in the art.

The phrase “codon optimization” refers to the selection of appropriate DNA nucleotides for use within a structural gene or fragment thereof that approaches codon usage within the organism of interest, and/or to a process of modifying a nucleic acid sequence for enhanced expression in the host cell of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Accordingly, genes can be tailored for optimal gene expression in a given organism based on codon optimization. Therefore, an optimized gene or nucleic acid sequence refers to a gene in which the nucleotide sequence of a native or naturally occurring gene has been modified in order to utilize statistically preferred or statistically-favored codons within the organism. The present invention explicitly encompasses polynucleotides encoding an enzyme of interest as disclosed herein which are codon optimized for expression in a yeast to be transformed according to the present invention, e.g., in Saccharomyces cerevisiae.

The term “regulatory elements” or “regulatory sequences” are used herein to describe DNA sequences which control the expression (transcription) of coding sequences, such as promoters and terminators. The regulatory elements used herein are suitable for use in a yeast to be transformed according to the present invention (e.g., in Saccharomyces cerevisiae) namely, capable of directing gene expression in the yeast.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence.

Promoters may be derived in their entirety from a native source, or be composed of different elements derived from different promoters found in nature, or even comprise synthetic nucleotide segments. Promoters can be constitutive (i.e., promoter activation is not regulated by an inducing agent and hence rate of transcription is constant), or inducible (i.e., promoter activation is regulated by an inducing agent). In most cases the exact boundaries of regulatory sequences have not been completely defined, and in some cases cannot be completely defined, and thus DNA sequences of some variation may have identical promoter activity.

The term “terminator” is directed to another regulatory DNA sequence which regulates transcription termination. A terminator sequence is operably linked to the 3′ terminus of the nucleic acid sequence to be transcribed.

The term “operably linked” means that a selected nucleic acid sequence is in proximity with a regulatory element (promoter or terminator) to allow the regulatory element to regulate expression of the selected nucleic acid sequence.

A plurality of expression cassettes may be assembled together into a single nucleic acid construct. Expression cassettes/nucleic acid constructs are further constructed into vectors enabling transformation into the host cells.

Expression cassettes, constructs and vectors may be assembled by a variety of different methods, including conventional molecular biology methods such as polymerase chain reaction (PCR), restriction endonuclease digestion, in vitro and in vivo assembly methods, as well as gene synthesis methods, or a combination thereof. Exemplary expression cassettes, constructs and vectors, and methods for their construction, are provided below.

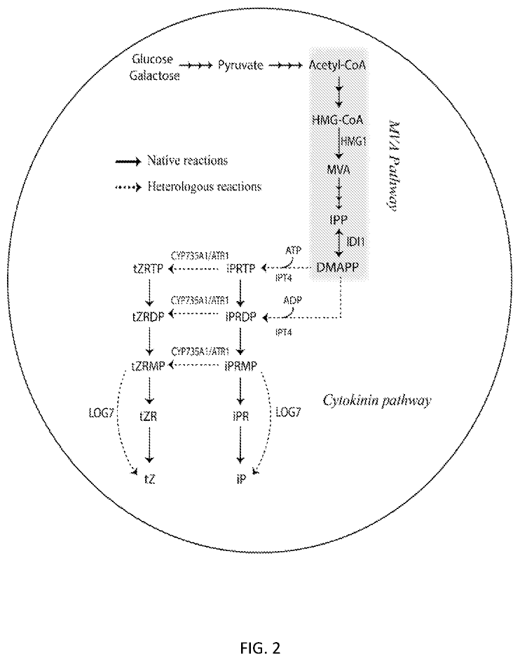

In some embodiments of the present invention, there is provided a design of heterologous cytokinin pathway in S. cerevisiae. The overall sequence of events in said pathway is as follows: Hexoses such as glucose and galactose are metabolized to pyruvate by the glycolytic pathway, which is then transformed to cytosolic acetyl-CoA in three sequential reactions. Acetyl-CoA is converted to isopentenyl diphosphate (IPP) and its isomer DMAPP by the endogenous mevalonate pathway (MVA). DMAPP and ATP/ADP are then incorporated to the cytokinin pathway by the expression of heterologous genes: IPT4, CYP735A1, ATR1, and LOG7 (dashed arrows, FIG. 2). Heterologous steps are complemented by native hydrolytic reactions of yeast nucleotide metabolism (solid arrows, FIG. 2) to complete the cytokinin pathway.

A strong cytokinin pathway flux could dramatically deplete the total adenine nucleotides pool. In order to avoid possible negative impact on strain fitness and process robustness, expression of the cytokinin biosynthetic genes under the control of inducible promoters is currently preferred. With this approach, the yeast can achieve enough biomass before producing the cytokinins, reducing the metabolic burden in the growth phase. In some exemplary embodiments, an inducible promoter suitable for use according to the present invention is a galactose-inducible promoter. Additional inducible promoters that may be used include, for example: copper-inducible CUP1 in S. cerevisiae, methanol-inducible AOX1 in P. Pastoris, and erythritol-inducible EYD1/EYK1 or fatty acid-inducible POX2 in Y. lipolytica. Each possibility represents a separate embodiment of the present invention.

### Vectors and cloning

The following sections describe exemplary vectors and cloning for generating cytokinin-producing S. cerevisiae strains according to some embodiments of the present invention. The cytokinin-producing S. cerevisiae strains described below express the following A. thaliana enzymes: IPT4, CYP735A1, ATR1 and LOG7. Further described are exemplary vectors and cloning for generating cytokinin-producing S. cerevisiae strains that express amino-terminal truncated versions of HMG-COA reductase lacking the transmembrane domain (a truncated version of the endogenous S. cerevisiae HMG-COA reductase and a truncated version of the HMG-COA reductase of Xanthophyllomyces dendrorhous) and over-express the endogenous S. cerevisiae isopentenyl isomerase (IDI1). Further described are exemplary vectors and cloning for generating cytokinin-producing S. cerevisiae strains that also express additional copies of CYP735A1, ATR1, and LOG7.

The amino acid sequence of IPT4 is set forth as SEQ ID NO: 1 (UniProt accession no. Q9SB60). The amino acid sequence of CYP735A1 is set forth as SEQ ID NO: 2 (UniProt accession no. Q9FF18). The amino acid sequence of ATR1 is set forth as SEQ ID NO: 3 (UniProt accession no. Q9SB48). The amino acid sequence of LOG7 is set forth as SEQ ID NO: 4 (UniProt accession no. Q8GW29). The amino acid sequence of S. cerevisiae truncate-HMG1 (Sc-tHMG1) is set forth as SEQ ID NO: 5 (Partial sequence of UniProt accession no. P12683). The amino acid sequence of Xanthophyllomyces dendrorhous truncated-HMG1 (Xd-tHMG1) is set forth as SEQ ID NO: 6 (Partial sequence of UniProt accession no. A0A5B8KS46). The amino acid sequence of S. cerevisiae IDI1 is set forth as SEQ ID NO: 7 (UniProt accession no. P15496). Table 1 shows the amino acid and gene sequences used in this invention.

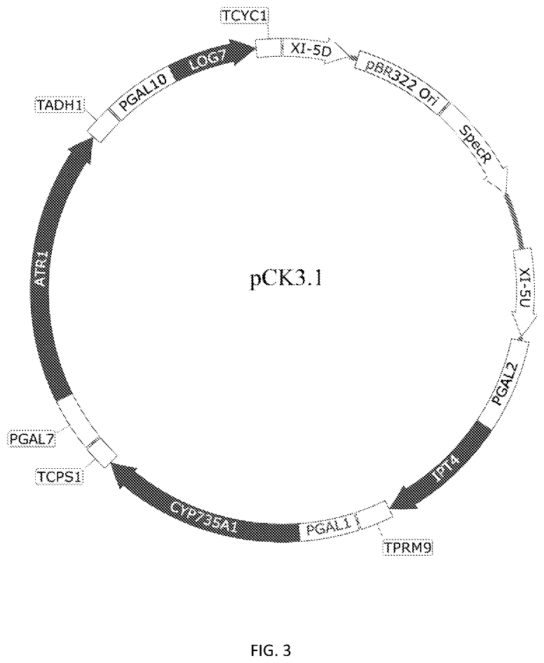

DNA polynucleotide sequences encoding the enzymes, codon-optimized for expression in S. cerevisiae (SEQ ID NO 8-14), were assembled in integrative yeast expression vectors. For this purpose, expression vectors were built employing Loop assembly method (Pollak et al., 2019, New Phytol., 222, 628-640; Pollak et al., 2020, Synth. Biol., 5 (1): ysaa001), which is based in Golden Gate cloning technique. The workflow included sequential assembly of DNA modules in different vectors termed Level 1 (L1), Level 2 (L2), and Level 3 (L3). Using this workflow, the polynucleotide sequences encoding the four enzymes were first cloned into entry vectors by the Gibson Assembly method. Then, transcriptional units for each gene (promoter-gene-terminator) were assembled into L1 vectors. For synthesizing L2 vectors, L1 units were joined together to arrange the whole biosynthetic pathway in a single construct. Finally, in L3 vectors, the L2 constructs were flanked between genomic integrating sequences. These L3 vectors were the final constructs used to transform the yeast cells and generate the cytokinin-producing strains. FIG. 3 illustrates an example map of L3 vectors. Table 2 shows the list and description of the constructed vectors, while Appendix I shows the sequences of the functional constructs which were assembled into these vectors.

TABLE 1
List of genes and polynucleotides used to construct S. cerevisiae
cytokinin-producing strains and corresponding SEQ ID NOs

| SEQ ID | |
|---|---|
| NO. | Description |
| 1 | A. thaliana IPT4 amino acid sequence |
| 2 | A. thaliana CYP735A1 amino acid sequence |
| 3 | A. thaliana ATR1 amino acid sequence |
| 4 | A. thaliana LOG7 amino acid sequence |
| 5 | S. cerevisiae truncated-HMG1 (Sc-tHMG1) amino acid |
| | sequence |
| 6 | X. dendrorhous truncated-HMG1 (Xd-tHMG1) amino acid |
| | sequence |
| 7 | S. cerevisiae IDI1 amino acid sequence |
| 8 | Codon-optimized nucleic acid sequence encoding A. thaliana |
| | IPT4 |
| 9 | Codon-optimized nucleic acid sequence encoding A. thaliana |
| | CYP735A1 |
| 10 | Codon-optimized nucleic acid sequence encoding A. thaliana |
| | ATR1 |
| 11 | Codon-optimized nucleic acid sequence encoding A. thaliana |
| | LOG7 |
| 12 | Nucleic acid sequence encoding S. cerevisiae tHMG1 |
| | (Sc-tHMG1) |
| 13 | Nucleic acid sequence encoding X. dendrorhous tHMG1 |
| | (Xd-tHMG1) |
| 14 | Nucleic acid sequence encoding S. cerevisiae IDI1 |

TABLE 2
Vectors used for the construction of cytokinin pathway and mevalonate
pathway (MVA) enhancer constructs and corresponding SEQ ID NOs

| Vector | SEQ ID | Loop | | |
|---|---|---|---|---|
| Name | NO. | Level | Functional construct | Description |
| pCK1.1 | 15 | 1 | P_{GAL2}_{_{—}}IPT4_T_{PRM9} | Transcriptional |
| pCK1.2 | 16 | 1 | P_{GAL1}_{_{—}}CYP735A1_T_{CPS1} | units of cytokinin |
| pCK1.3 | 17 | 1 | P_{GAL7}_{_{—}}ATR1_T_{ADH1} | pathway genes |
| pCK1.4 | 18 | 1 | P_{GAL10}_{_{—}}LOG7_T_{CYC1} | using Galactose- |
| | | | | inducible |
| | | | | promoters |
| pMVA1.1 | 19 | 1 | P_{TDH3}_{_{—}}Sc-tHMG1_T_{PRM9} | Transcriptional |
| pMVA1.2 | 20 | 1 | P_{TEF1}_{_{—}}Xd-tHMG1_T_{ADH1} | units of MVA |
| pMVA1.3 | 21 | 1 | P_{PGK1}_{_{—}}IDI1_T_{CPS1} | pathway genes |
| | | | | using constitutive |
| | | | | promoters |
| pMVA1.4 | 22 | 1 | P_{GAL2}_{_{—}}Sc-tHMG1_T_{PRM9} | Transcriptional |
| pMVA1.5 | 23 | 1 | P_{GAL7}_{_{—}}Xd-tHMG1_T_{ADH1} | units of MVA |
| pMVA1.6 | 24 | 1 | P_{GAL1}_{_{—}}IDI1_T_{CPS1} | pathway genes |
| | | | | using Galactose- |
| | | | | inducible |
| | | | | promoters |
| pCK2.1 | 25 | 2 | CK1.1/CK1.2/CK1.3/CK1.4 | Cytokinin pathway |
| | | | | using Galactose- |
| | | | | inducible |
| | | | | expression |
| pCK2.2 | 26 | 2 | CK1.2/CK.1.3/CK.1.4 | Partial cytokinin |
| | | | | pathway using |
| | | | | Galactose- |
| | | | | inducible |
| | | | | expression |
| pMVA2.1 | 27 | 2 | MVA1.1/MVA1.3/MVA1.2 | MVA enhancer |
| | | | | construct using |
| | | | | constitutive |
| | | | | expression |
| pMVA2.2 | 28 | 2 | MVA1.4/MVA1.6/MVA1.5 | MVA enhancer |
| | | | | construct using |
| | | | | Galactose- |
| | | | | inducible |
| | | | | expression |
| pCK3.1 | 29 | 2 | XI-5U/CK2.1/XI-5D | Inducible cytokinin |
| | | | | pathway flanked by |
| | | | | yeast integrating |
| | | | | sequences |
| pCK3.2 | 30 | 2 | XI-3U/CK2.2/XI-3D | Inducible partial |
| | | | | cytokinin pathway |
| | | | | flanked by yeast |
| | | | | integrating |
| | | | | sequences |
| pMVA3.1 | 31 | 2 | X-2U/MVA3.1/X-2D | Constitutive MVA |
| | | | | enhancer construct |
| | | | | flanked by yeast |
| | | | | integrating |
| | | | | sequences |
| pMVA3.2 | 32 | 2 | X-2U/MVA3.2/XI-2D | Inducible MVA |
| | | | | enhancer construct |
| | | | | flanked by yeast |
| | | | | integrating |
| | | | | sequences |

Table 2: Each transcriptional unit (functional construct) is composed of a promoter, a gene, and a terminator (P_gene_T). Genomic integration sequences are denoted by chromosome and the relative number of the site, according to Mikkelsen et al., 2012, Metab. Eng., 14, 104-111. For example, XI-5: chromosome XI, site 5, divided into Up site (XI-5U) and Down site (XI-D).

### Construction of Cytokinin-Producing Strains of S. cerevisiae

The L3 vectors were transformed into the parent yeast strain CEN.PK113-5D (genotype MATa ura3-52 HIS3, LEU2 TRP1 MAL2-8c SUC2) by CRISPR/Cas9 technology (Jakočiunas et al., 2016, Metab. Eng., 34:44-59; Shaw and Ellis, 2017, Quick and easy CRISPR engineering in Saccharomyces cerevisiae. This system requires incorporating three vectors into the yeast cells: a Cas9 containing vector, a guide RNA (gRNA) containing vector, and the cytokinin pathway L3 integrating vector. The three vectors were linearized by enzyme digestion and transformed by LiAc/PEG/ssDNA method (Gietz, 2014, Yeast transformation by the LiAc/SS carrier DNA/PEG method, in: Yeast Genetics. Humana Press, New York, pp. 1-12). Cas9 and sgRNA together carried out the double-strand break into the specific integrating site of the yeast genome. The latter facilitates the integration of the cytokinin pathway construct by homologous recombination in this specific site.

The transformants were selected in complete synthetic media without uracil (SC-URA). The isolation of the transformants that integrated the constructs were performed by genomic PCR of several colonies. These PCRs were carried out using primers which amplified regions between integration sites and different inner sites of the constructs (e.g., promoters and terminators). Three to five colonies of each constructed strain were selected and cultivated in shake flasks to test cytokinin production. Recycling of the selection marker was performed by serial cultivation of the strains in YPD until the loss of Cas9 episomal vectors, which carried the URA3 gene. Colonies that fail to grow in SC-URA were used for the next transformation rounds.

Table 3 summarizes the four constructed strains. CK1 strain contains the whole cytokinin pathway (IPT4, CYP735A1, ATR1, and LOG1) arranged in tandem transcriptional units and integrated at XI-5 site (chromosome XI, site 5). The four transcriptional units were placed under the control of galactose-inducible promoters. Different promoters and terminators were used for each transcriptional unit in order to avoid homologous recombination and ensure strain stability. CK1 was used as parent strain to construct CK2.1 by the integration of MVA enhancement construct in X-2 site. This construct consisted in two copies of tHMG1 gene (one from S. cerevisiae and other from X. dendrorhous) and one copy of IDI1 gene, all under the control of strong constitutive promoters. Similarly, CK.2.2 strains were built to enhance MVA pathway in CK1, but in this case, using a galactose-inducible version of the MVA enhancement construct. Finally, CK3.1 strain was generated by the transformation of CK2.1 with a construct that expressed additional copies of CYP735A1, ATR1, and LOG7, also under galactose-inducible promoters.

TABLE 3
S cerevisiae strains constructed for the heterologous production of cytokinins

| | Parent | Transformed | | |
|---|---|---|---|---|
| Name | strain | Vector | Genotype | Comments |
| CK1 | CEN.PK11 | pCK3.1 | XI-5Δ:: P_{GAL2}_{_{—}}IPT4_T_{PRM9} | Galactose- |
| | 3-5D | | P_{GAL1}_{_{—}}CYP735A1_T_{CPS1} | inducible |
| | | | P_{GAL7}_{_{—}}ATR1_T_{ADH1} | expression of |
| | | | P_{GAL10}_{_{—}}LOG7_T_{CYC1} | cytokinin |
| | | | | pathway |
| CK2.1 | CK1 | pMVA3.1 | X-2Δ:: P_{TDH3}_{_{—}}Sc- | Enhancement of |
| | | | tHMG1_T_{PRM9} | MVA pathway |
| | | | P_{PGK1}_{_{—}}IDI1_T_{CPS1} | (constitutive) for |
| | | | P_{TEF1}_{_{—}}Xd-tHMG1_T_{ADH1} | increased |
| | | | | cytokinin |
| | | | | biosynthesis |
| CK2.2 | CK1 | pMVA3.2 | X-2Δ:: P_{GAL2}_{_{—}}Sc- | Enhancement of |
| | | | tHMG1_T_{PRM9} | MVA pathway |
| | | | P_{GAL1}_{_{—}}IDI1_T_{CPS1} | (galactose- |
| | | | P_{GAL7}_{_{—}}Xd-tHMG1_T_{ADH1} | inducible) for |
| | | | | increased |
| | | | | cytokinin |
| | | | | biosynthesis |
| CK3.1 | CK2.1 | pCK3.2 | XI-3Δ:: | Expression of |
| | | | _{GAL1}_{_{—}}CYP735A1_T_{CPS1} | additional copies |
| | | | P_{GAL7}_{_{—}}ATR1_T_{ADH1} | of three genes of |
| | | | P_{GAL10}_{_{—}}LOG7_T_{CYC1} | the pathway to |
| | | | | change |
| | | | | cytokinins |
| | | | | proportions |

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Cytokinin Production of CK1 Strain in Shake Flasks

By preliminary analysis of several transformants of CK1 strain generated as described above, two clearly differentiated cytokinin-producing phenotypes were detected, named as CK1-A and CK1-B. The sub-strains were grown in baffled shake flasks, containing YPDG medium (1% yeast extract, 2% peptone, 1% glucose, and 1% galactose) at 30° C. and 160 rpm. The cultures were sampled at 48 h and the fermented culture media was analyzed by HPLC-DAD. This method quantifies trans-zeatin (tZ), trans-zeatin riboside ((ZR), isopentenyladenine (iP), and isopentenyladenine riboside (iPR). The method is summarized as follow:
Column: Phenomenex Luna® 5 μm C18 (2) 100 A (250×4.6 mm)
Mobile phase A: Water
Mobile phase B: Acetonitrile.
Column oven temperature: 35° C.
Detection wavelength: 254 nm.
Gradient (min: % A): 0:85, 8:85, 25:70, 30:70, 40:85, 60:85.

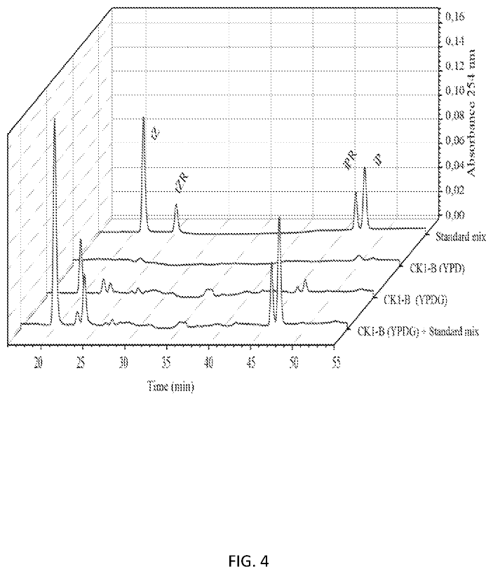

Exemplary HPLC-DAD analysis of fermented medium from CK1-B sub-strain in the presence or absence of induction is shown in FIG. 4. The top chromatogram shows the separation of a standard mixture of trans-zeatin (tZ, 21 min), trans-zeatin riboside (tZR. 24 min), isopentenyladenine riboside (iPR, 46 min), and isopentenyladenine (iP, 47 min). The fermented medium of CK1-B in the absence of induction (YPD medium) did not show peaks at the cytokinin retention times (second chromatogram from the top). By contrast, production of the four cytokinins was observed after the induction of the strain in YPDG medium (third chromatogram from the top). The same sample was spiked with the standard mixture to validate the compound's identities (bottom chromatogram).

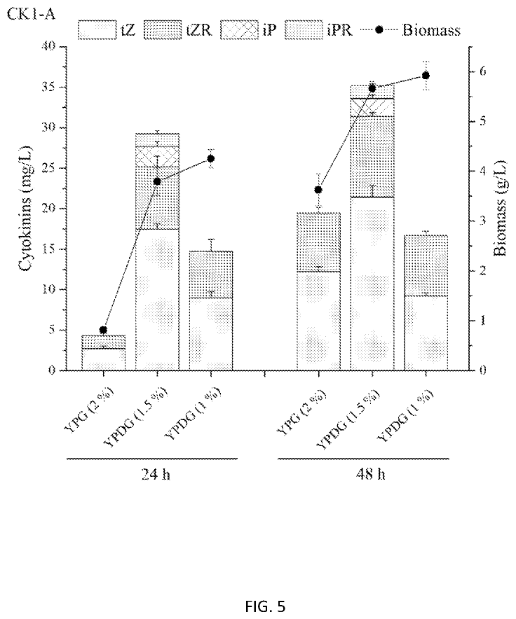

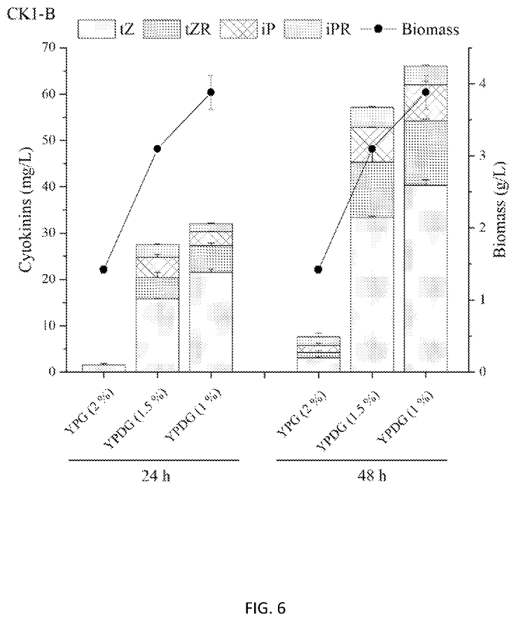

For a more detailed analysis of both phenotypes CK1-A and CK1-B, three independent transformants of each strain were cultivated in triplicate in baffled shake flasks, containing YPDG media with different glucose/galactose ratios. The cultures were sampled at 24 and 48 h (FIG. 5 and FIG. 6). Yeast cells consume glucose before galactose, enabling a growth phase without inducing the heterologous pathway (Nguyen-Huu et al., 2015, PLOS Comput. Biol., 11: e1004399; van den Brink et al., 2009, Microbiology, 155:1340-1350). Once the glucose is depleted, galactose is incorporated and consumed. At this stage, the inducible Gal promoters trigger the expression of the pathway, and the production of cytokinins starts.

Overall, CK-1B produced higher concentrations of total cytokinins than CK1-A. The strain production varied in response to the different induction strategies (namely, different ratios of glucose/galactose in the culture medium). As seen in FIG. 6, CK-1B reached up to 66 mg/L of total cytokinins after 48 hours of cultivation in YPDG 1% (1% glucose, 1% galactose), with tZ as the predominant compound (40.3 mg/L). The production of cytokinins and biomass by CK-1B were slightly lower in YPDG 1.5%, but dramatically reduced in YPG 2% culture medium. The cytokinin pathway uses ATP and DMAPP as main precursors. The biosynthesis of these molecules involves high energy requirements for the cell (Tarkowská and Strnad, 2018, Planta, 247:1051-1066). Without being bound by any particular theory of a mechanism of action, the metabolic burden generated by the cytokinin production could explain why the conservative induction strategy was more efficient in the transformed strains. With this approach, the yeast can achieve enough biomass before initiating the production of the cytokinins, reducing the metabolic burden in the growth phase. As seen in FIG. 5 CK1-A showed a lower production rate than CK-1B but withstood better the more aggressive induction strategy (YPG 2%).

### Example 2—Cytokinin Production of CK1 Strain in 1 L Batch Bioreactor

In order to characterize the kinetics of the CK-1B strain, a batch fermentation was carried out in benchtop bioreactor. The fermentation was performed in 700 mL of YPDG medium containing 2% peptone, 1.5% yeast extract, 1.5% of glucose, and 1.5% galactose. A SIMATIC PCS7 control system was used to monitor and control the cultivations at 30° C., pH=5.0, and dissolved oxygen above 2.8 mg/L. After 32 hours fermentation, the culture was supplemented with a pulse of 2% galactose. Culture samples were periodically collected for quantification of biomass, sugars, ethanol, and cytokinins.

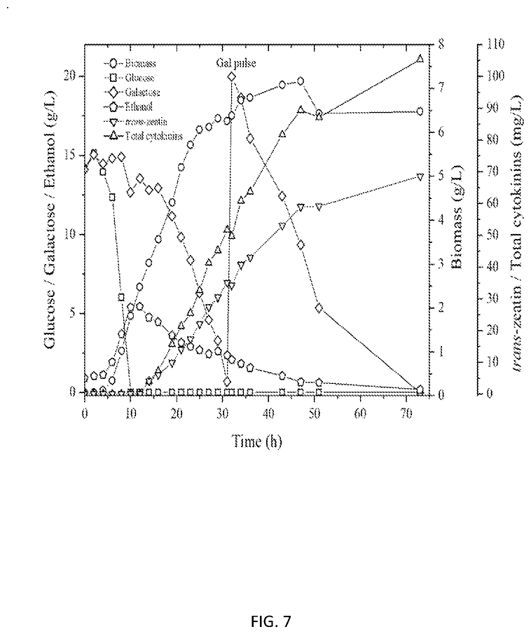

The results are presented in FIG. 7. As expected, the strain started consuming glucose without galactose utilization. At this growth stage, biomass accumulated without producing cytokinins. Once glucose was exhausted, the yeast began to metabolize the galactose as a carbon source, the heterologous biosynthetic pathway was induced, and the cells started to produce cytokinins. This kinetics confirmed that the inducible system is tightly regulated by the carbon source. The maximum specific growth rate (μₘₐₓ) was 0.46 h⁻¹ for glucose-consuming and 0.07 h⁻¹ for the galactose (induction) phase. It is worthy to note that the μₘₐₓ of the parent strain grown in galactose was 0.17 h⁻¹ (Bro et al., 2005, Appl. Environ. Microbiol., 71:6465-6472), corroborating that the induction of the cytokinin pathway results in a strong metabolic burden for the induced cells. Interestingly, due to the low specific growth rate (below the critical growth rate), the strain did not produce ethanol. However, during the induction phase, the strain metabolized the ethanol produced during the glucose consumption phase, simultaneously with the galactose.

Cytokinin production parameters achieved in this fermentation are presented in Table 4. The strain CK1-B produced up to 94.6 mg/L of total cytokinins after 73 hours of fermentation with predominance of tZ (68.6 mg/L). The volumetric productivities were 1.67 mg/L/h of total cytokinins and 1.13 mg/L/h of tZ.

TABLE 4
Cytokinin production parameters for
the batch fermentation of CK1 strain

| | | | | Specific |
|---|---|---|---|---|
| | Concen- | Yield | Volumetric | production |
| | tration | (mg/g- | productivity | rate (mg/g- |
| Compound | (mg/L) | biomass) | (mg/L/h) | biomass/h) |
| tZ | 68.6 | 10.6 | 1.13 | 0.18 |
| Total CK | 105.4 | 16.2 | 1.67 | 0.27 |

Table 4: The maximum concentrations and yields were obtained at 73 hours of fermentation. Volumetric productivities and specific production rates were calculated at 31 hours (before the galactose pulse). tZ: trans-zeatin. Total CK: total cytokinins (trans-zeatin, trans-zeatin riboside, isopentenyladenine, and isopentenyladenine riboside).

### Example 3—Cytokinin Production of CK2 and CK3 Strains in Shake Flasks

The carbon flux through the native MVA pathway could be restricting the DMAPP availability and consequently limiting the cytokinin biosynthesis in CK1 strains. To increase MVA pathway flux, two copies of tHMG1 gene were overexpressed. Additionally, IDI1 gene was overexpressed to enhances IPP/DMAPP isomerization. The expression of these genes was carried out by the integration of the MVA enhancement construct (Table 3) in both constitutive and galactose-inducible variants to generate the strains CK2.1 and CK2.2, respectively. The strains were cultivated in triplicate in baffled shake flasks containing YPDG (1% glucose, 1% galactose).

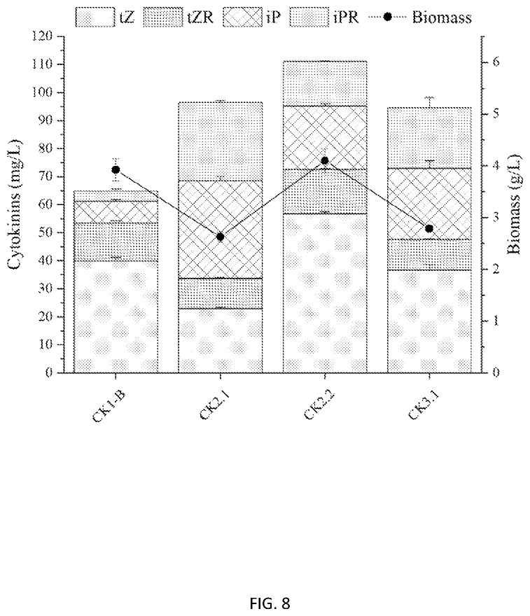

The results are shown in FIG. 8. The integration and expression of constitutive MVA enhancement construct (CK2.1 strain) yielded a 1.5-fold increase in total cytokinin production (96.4 mg/L) with respect to the parent strain CK1 (64.9 mg/L). In comparison to CK1-B, a redistribution of cytokinin proportions was observed in CK2.1, with a 1.7-fold decrease in tZ and 4.5-fold-increase in iP concentrations. The CK2.2 strain, which contains the galactose-inducible version of the MVA enhancement construct, produced 111.1 mg/L of total cytokinins. This strain performed slightly better than CK2.1 and showed a higher tZ/iP ratio, more similar to the proportions observed in the CK1-B parent strain. These results suggest that the MVA pathway flux partially limits the heterologous cytokinin biosynthesis in yeast.

As an example of how cytokinin proportion can be redistributed by the expression of additional copies of the cytokinin pathway, a construct harboring CYP735A, ATR1 and LOG7 was integrated in CK2.1 strain. The resulting strain, CK3.1, showed a 1.6-fold increase in tZ and a 1.3-fold decrease iP concentrations. The expression of an extra copy of cytokinin hydroxylase system (CYP735A/ATR1) without extra expression of IPT4 redirected the flux to hydroxylated cytokinins. This result illustrates how the pathway can be adjusted in order to control the cytokinin ratios depending on the desired application of the product.

### Example 4—Cytokinin Production of CK2.2 Strain in Fed-Batch Fermentation

To further increase cytokinin production, the highest producing strain CK2.2 was scaled to high-density cultures in 1 L bioreactors. A SIMATIC PCS7 control system was used to monitor and control the cultivations at 30° C., pH=5.0, and dissolved oxygen above 2.8 mg/L. Ammonium hydroxide (25% m/m) was used to automatically control the pH. Excessive foam formation was prevented by the use of liquid silicone 10% v/v when required. The dissolved oxygen was maintained above 2.6 mg/L by an automatic algorithm which controls agitation (160-280 rpm), air flux, and pure oxygen flux. The total gas flux was maintained between 0.4-0.5 L/min, varying the percent of air and pure oxygen depending on the culture demand. Gas outflux was measured using a Blue Vary gas sensor (BlueSens). The media employed for the fermentation are detailed in Table 5. The growth medium was used to obtain high cell densities while the induction medium was applied to induce the production of cytokinins after the growth phase.

TABLE 5
Media composition used for the fed-batch
fermentation of cytokinins production

| | | |
|---|---|---|
| Component | Growth Medium | Induction Medium |

| Glucose | 450 | g/L | 0 | |
|---|---|---|---|---|
| Galactose | 0 | | 450 | g/L |
| Yeast extract | 5 | g/L | 0 | |
| Casamino acids | 30 | g/L | 15 | g/L |
| Vitamin solution | 15 | mL/L | 15 | mL/L |
| Trace solution | 9 | mL/L | 9 | mL/L |
| Uracil | 3 | g/L | 2 | g/L |

| | | |
|---|---|---|
| CaCl₂*2H₂0 | 1.06 | 1.06 |
| FeSO₄*7H₂O | 0.22 | 0.22 |
| MgSO₄*7H₂O | 5.5 | 5.5 |
| KH₄PO₄ | 15 | 15 |

Table 5: The detailed composition of vitamin and trace solution together with the protocol for the preparation of the media can be found at López et al., 2019, Front. Bioeng. Biotechnol., 7: 171)

The bioreactor was inoculated with 300 mL of shake flask cultures of CK2.2 strain.

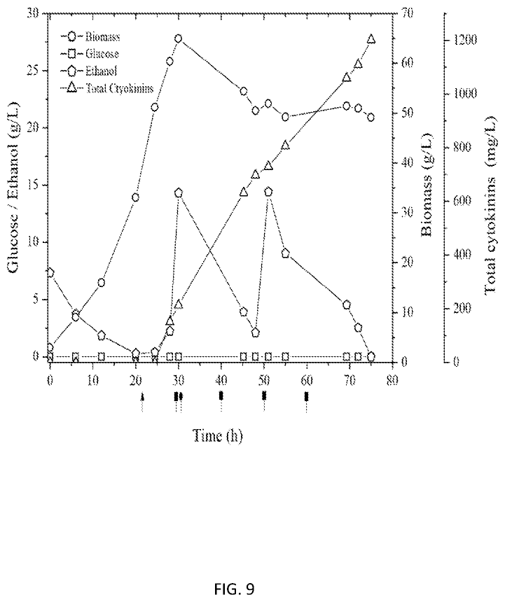

The growth phase strategy consisted in a glucose-limited exponential feeding using the growth medium (Table 5). The growth rate was maintained at μ=0.12 h⁻¹. In order to adapt the cells to galactose before the induction, a pulse of 15 g/L of galactose was administered at 22 h of the growth phase. The growth medium was replaced by the induction medium at 30 h of fermentation, and the latter was administered at a constant feeding rate of 0.14 mL/min. In order to increase the supply of cytosolic acetyl-CoA and boost cytokinin production, periodic pulses of 15 g/L of ethanol were supplied every 10 h during the whole induction phase (FIG. 9).

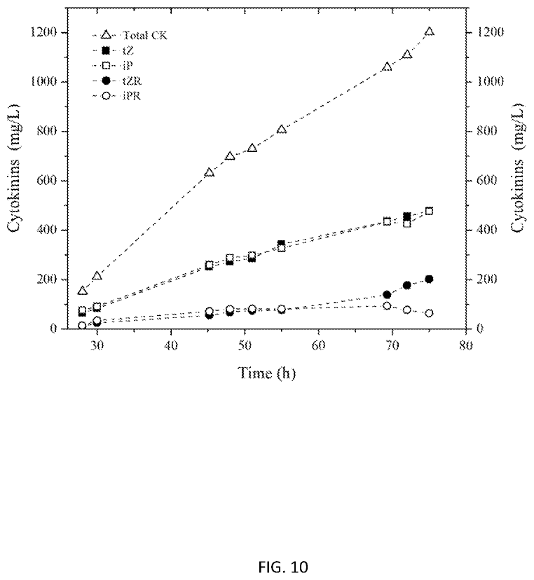

The culture reached 65 g/L at the end of the growth phase (30 h). Yeast cells started to produce cytokinins at 28 h, six hours after the galactose adaptation pulse. This adaptation strategy facilitated the transition from the growth medium to the induction medium and speeded up the production of cytokinins. The fed-batch process reached 1.2 g/L of total cytokinins in the culture medium after 75 h of fermentation (FIG. 9), with a productivity of 16 mg/L/h. The production kinetics of each cytokinin (tZ, tZR, iP and iPR) is shown in FIG. 10. In addition to the cytokinins secreted to the culture media, the strain also accumulated 308 mg/L of total cytokinins in the biomass. The total cytokinin concentration (culture medium+biomass) reached 1.5 g/L with a productivity of 20.1 mg/L/h.

Table 6 presents the final cytokinin composition in the culture medium and biomass after 75 h of fermentation. Table 7 shows the detailed production parameters.

TABLE 6
Final cytokinins concentrations in culture medium and biomass
after 75-hour fed-batch fermentation of CK2.2 strain

| | tZ | tZR | iP | iPR | Total CK |
|---|---|---|---|---|---|
| Culture medium | 477.6 | 201.9 | 476.4 | 63.5 | 1201.3 |
| Biomass | 131.0 | 6.5 | 125.3 | 8.5 | 308.3 |
| Total (medium + Biomass) | 608.6 | 208.4 | 601.7 | 72.0 | 1509.6 |

Table 6: tZ: trans-zeatin. tZR: trans-zeatin riboside. iP: isopentenyladenine. iPR: isopentenyladenine riboside. Total CK: total cytokinins.

TABLE 7
Total cytokinin production parameters for the fed-
batch fermentation of CK2.2 strain. All parameters
were calculated at 75 hours of fermentation

| | | | | Specific |
|---|---|---|---|---|
| | Concen- | Yield | Volumetric | production |
| | tration | (mg/g- | productivity | rate (mg/g- |
| | (mg/L) | biomass) | (mg/L/h) | biomass/h |
| Culture medium | 1201.3 | 24.5 | 16.0 | 0.33 |
| Biomass | 308.3 | 6.3 | 4.1 | 0.08 |
| Total (medium + | 1509.6 | 30.7 | 20.1 | 0.41 |
| Biomass) | | | | |

### Example 5—Biological Activity of the Yeast-Derived Cytokinins

The bioactivity of the produced cytokinins was tested in A. thaliana by hypocotyl length assay. This assay is based on the capacity of cytokinins to inhibit hypocotyl elongation in A. thaliana seedlings (Cary et al., 1995, Plant Physiol., 107 (4): 1075-1082). Seeds of A. thaliana Col-0 were surface sterilized, sown on 0.5× Murashige and Skoog (MS) medium (Murashige and Skoog, 1962, Physiol. Plant., 15(13):473-497) (0.7% agar) without sucrose, and stratified in darkness for 3 days at 4° C. Seedlings were grown in complete darkness in a growth cabinet at 21°° C. Hypocotyl lengths were determined after 8 days of growth, and at least 15 seedlings were measured for each treatment. The culture medium obtained by the fed-batch fermentation of CK2.2 strain (Example 4) was diluted 1/10000 to a final concentration 0.12 mg/L of total cytokinins for the assay. Trans-zeatin chemical standard was used as positive control and fermented YPDG medium of CEN.PK113-5D strain was used as a negative control

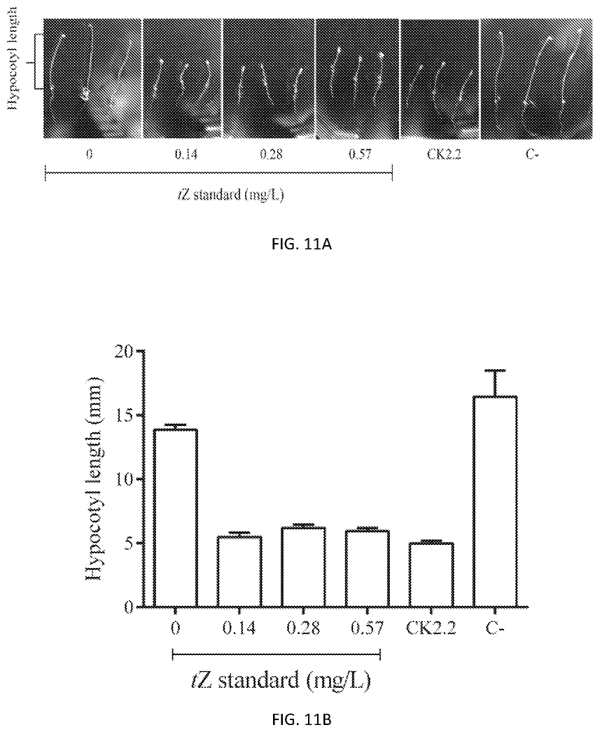

FIG. 11 shows the results of the bioassays. All the treatments with tZ standard (0.14, 0.28, and 0.56 mg/L) showed a mean reduction of 58% in hypocotyl lengths with no significant difference between them. The diluted culture medium of CK2.2 fed-batch fermentation triggered a similar effect (64%) to that of tZ standards. The culture medium from the parent strain CEN.PK113-5D (C−) did not show significant differences with the blank (0 mg/L of tZ), indicating absence of bioactivity. These results demonstrate the strong bioactivity of the cytokinins mixture obtained by the yeast fermentation process.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A genetically-modified yeast producing at least one cytokinin selected from the group consisting of trans-zeatin, trans-zeatin riboside, isopentenyladenine, isopentenyladenine riboside and any combination thereof, the genetically modified yeast comprises a plurality of exogenous polynucleotides comprising:
(i) a polynucleotide encoding an isopentenyl transferase;
(ii) a polynucleotide encoding a cytokinin hydroxylase;
(iii) a polynucleotide encoding an NADPH-cytochrome P450 reductase; and
(iv) a polynucleotide encoding a cytokinin riboside 5′-monophosphate phosphoribohydrolase.

2. The genetically-modified yeast of claim 1, wherein the encoded enzymes are selected from the group consisting of plant enzymes, fungal enzymes, homologs thereof and any combination thereof.

3. The genetically-modified yeast of claim 2, wherein the encoded enzymes are plant enzymes or homologs thereof.

4. The genetically-modified yeast of claim 3, wherein the plant is Arabidopsis thaliana.

5. The genetically-modified yeast of any one of claims 3-4, wherein the isopentenyl transferase comprises an amino acid sequence having at least 80% identity to the amino acid sequence of A. thaliana IPT4.

6. The genetically-modified yeast of claim 5, wherein the A. thaliana IPT4 comprises the amino acid sequence set forth in SEQ ID NO: 1.

7. The genetically-modified yeast of any one of claims 3-6, wherein the cytokinin hydroxylase comprises an amino acid sequence having at least 80% identity to the amino acid sequence of A. thaliana CYP735A1.

8. The genetically-modified yeast of claim 7, wherein the A. thaliana CYP735A1 comprises the amino acid sequence set forth in SEQ ID NO: 2.

9. The genetically-modified yeast of any one of claims 3-8, wherein the NADPH-cytochrome P450 reductase comprises an amino acid sequence having at least 80% identity to the amino acid sequence of A. thaliana ATR1.

10. The genetically-modified yeast of claim 9, wherein the A. thaliana ATR1 comprises the amino acid sequence set forth in SEQ ID NO: 3.

11. The genetically-modified yeast of any one of claims 3-10, wherein the cytokinin riboside 5′-monophosphate phosphoribohydrolase comprises an amino acid sequence having at least 80% identity to the amino acid sequence of A. thaliana LOG7.

12. The genetically-modified yeast of claim 11, wherein the A. thaliana LOG7 comprises the amino acid sequence set forth in SEQ ID NO: 4.

13. The genetically-modified yeast of any one of claims 3-12, comprising:
(i) an exogenous polynucleotide encoding an isopentenyl transferase having at least 80% identity to the amino acid sequence of A. thaliana IPT4;
(ii) an exogenous polynucleotide encoding a cytokinin hydroxylase having at least 80% identity to the amino acid sequence of A. thaliana CYP735A1;
(iii) an exogenous polynucleotide encoding an NADPH-cytochrome P450 reductase having at least 80% identity to the amino acid sequence of A. thaliana ATR1; and
(iv) an exogenous polynucleotide encoding a cytokinin riboside 5′-monophosphate phosphoribohydrolase having at least 80% identity to the amino acid sequence of A. thaliana LOG7.

14. The genetically-modified yeast of any one of claims 1-13, wherein the yeast is Saccharomyces cerevisiae.

15. The genetically-modified yeast of claim 14, comprising an exogenous polynucleotide encoding A. thaliana IPT4 comprising the nucleic acid sequence set forth in SEQ ID NO: 8.

16. The genetically-modified yeast of any one of claims 14-15, comprising an exogenous polynucleotide encoding A. thaliana CYP735A1 comprising the nucleic acid sequence set forth in SEQ ID NO: 9.

17. The genetically-modified yeast of any one of claims 14-16, comprising an exogenous polynucleotide encoding A. thaliana ATR1 comprising the nucleic acid sequence set forth in SEQ ID NO: 10.

18. The genetically-modified yeast of any one of claims 14-17, comprising an exogenous polynucleotide encoding A. thaliana LOG7 comprising the nucleic acid sequence set forth in SEQ ID NO: 11.

19. The genetically-modified yeast of claim 14, comprising: an exogenous polynucleotide encoding A. thaliana IPT4 comprising the nucleic acid sequence set forth in SEQ ID NO: 8; an exogenous polynucleotide encoding A. thaliana CYP735A1 comprising the nucleic acid sequence set forth in SEQ ID NO: 9; an exogenous polynucleotide encoding A. thaliana ATR1 comprising the nucleic acid sequence set forth in SEQ ID NO: 10; and an exogenous polynucleotide encoding A. thaliana LOG7 comprising the nucleic acid sequence set forth in SEQ ID NO: 11.

20. The genetically-modified yeast of any one of the claims 1-19, wherein each of the plurality of exogenous polynucleotides or a combination thereof is comprised within an expression cassette further comprising at least one regulatory element.

21. The genetically-modified yeast of claim 20, wherein the regulatory element is selected from a promoter, an enhancer, a termination sequence and any combination thereof.

22. The genetically modified yeast of claim 21, wherein the promoter is an inducible promoter.

23. The genetically-modified yeast of claim 22, wherein the promoter is a galactose-inducible promoter.

24. The genetically-modified yeast of any one of claims 14-23, comprising: an expression cassette encoding A. thaliana IPT4 comprising the nucleic acid sequence set forth in SEQ ID NO: 15; an expression cassette encoding A. thaliana CYP735A1 comprising the nucleic acid sequence set forth in SEQ ID NO: 16; an expression cassette encoding A. thaliana ATR1 comprising the nucleic acid sequence set forth in SEQ ID NO: 17; and an expression cassette encoding A. thaliana LOG7 comprising the nucleic acid sequence set forth in SEQ ID NO: 18.

25. The genetically-modified yeast of claim 24, wherein the expression cassettes are arranged in a single polynucleotide molecule.

26. The genetically-modified yeast of claim 25, wherein the single polynucleotide molecule comprises the nucleic acid sequence set forth in SEQ ID NO: 25.

27. The genetically-modified yeast of any one of claims 1-26, further comprising at least one additional exogenous polynucleotide selected from the group consisting of:
(A) one or more exogenous polynucleotide encoding an amino-terminal truncated HMG-COA reductase lacking the transmembrane domain; and
(B) an exogenous polynucleotide encoding an isopentenyl isomerase.

28. The genetically-modified yeast of claim 27, comprising at least one of: an exogenous polynucleotide encoding a truncated HMG-COA reductase having at least 80% identity to the amino acid sequence of an amino-terminal truncated HMG-COA reductase 1 of Saccharomyces cerevisiae (Sc-tHMG1); an exogenous polynucleotide encoding a truncated HMG-COA reductase having at least 80% identity to the amino acid sequence of Xanthophyllomyces dendrorhous amino-terminal truncated HMG-COA reductase 1 (Xd-tHMG1); or a combination thereof.

29. The genetically-modified yeast of claim 28, wherein the Sc-tHMG1 comprises the amino acid sequence set forth in SEQ ID NO: 5, and the Xd-tHMG1 comprises the amino acid sequence set forth in SEQ ID NO: 6.

30. The genetically-modified yeast of claim 28 or claim 29, wherein the isopentenyl isomerase is an isopentenyl isomerase having at least 80% identity to the amino acid sequence of S. cerevisiae IDI1.

31. The genetically-modified yeast of claim 30, wherein the S. cerevisiae IDI1 comprises the amino acid sequence set forth in SEQ ID NO: 7.

32. The genetically-modified yeast of claim 27, comprising:
(A) an exogenous polynucleotide encoding a truncated HMG-COA reductase having at least 80% identity to the amino acid sequence of Sc-tHMG1;
(B) an exogenous polynucleotide encoding a truncated HMG-COA reductase having at least 80% identity to the amino acid sequence of Xd-tHMG1; and
(C) an exogenous polynucleotide encoding an isopentenyl isomerase having at least 80% identity to the amino acid sequence of S. cerevisiae IDI1.

33. The genetically-modified yeast of any one of claims 27-32, wherein the yeast is Saccharomyces cerevisiae.

34. The genetically-modified yeast of claim 33, comprising an exogenous polynucleotide encoding Sc-tHMG1 comprising the nucleic acid sequence set forth in SEQ ID NO: 12.

35. The genetically-modified yeast of claim 33 or claim 34, comprising an exogenous polynucleotide encoding Xd-tHMG1 comprising the nucleic acid sequence set forth in SEQ ID NO: 13.

36. The genetically-modified yeast of any one of claims 33-35, comprising an exogenous polynucleotide encoding S. cerevisiae IDI1 comprising the nucleic acid sequence set forth in SEQ ID NO: 14.

37. The genetically-modified yeast of claim 33, comprising: an exogenous polynucleotide encoding Sc-tHMG1 comprising the nucleic acid sequence set forth in SEQ ID NO: 12; an exogenous polynucleotide encoding Xd-tHMG1 comprising the nucleic acid sequence set forth in SEQ ID NO: 13; and an exogenous polynucleotide encoding S. cerevisiae IDI1 comprising the nucleic acid sequence set forth in SEQ ID NO: 14.

38. The genetically-modified yeast of any one of claims 27-37, wherein each of the plurality of exogenous polynucleotides or a combination thereof is comprised within an expression cassette further comprising at least one regulatory element.

39. The genetically-modified yeast of claim 38, wherein the regulatory element is selected from a promoter, an enhancer, a termination sequence and any combination thereof.

40. The genetically-modified yeast of claim 39, wherein the promoter is an inducible promoter.

41. The genetically-modified yeast of claim 40, wherein the promoter is a galactose-inducible promoter.

42. The genetically-modified yeast of claim 41, comprising: an expression cassette encoding Sc-tHMG1 comprising the nucleic acid sequence set forth in SEQ ID NO: 22; an expression cassette encoding Xd-tHMG1 comprising the nucleic acid sequence set forth in SEQ ID NO: 23; and an expression cassette encoding S. cerevisiae IDI1 comprising the nucleic acid sequence set forth in SEQ ID NO: 24.

43. The genetically-modified yeast of claim 39, wherein the promoter is a constitutive promoter.

44. The genetically-modified yeast of claim 43, comprising: an expression cassette encoding Sc-tHMG1 comprising the nucleic acid sequence set forth in SEQ ID NO: 19; an expression cassette encoding Xd-tHMG1 comprising the nucleic acid sequence set forth in SEQ ID NO: 20; and an expression cassette encoding S. cerevisiae IDI1 comprising the nucleic acid sequence set forth in SEQ ID NO: 21.

45. The genetically-modified yeast of claim 42 or claim 44, wherein the expression cassettes are arranged in a single polynucleotide molecule.

46. The genetically-modified yeast of claim 45, wherein the single polynucleotide molecule comprises a nucleic acid sequence selected from SEQ ID NO: 27 and SEQ ID NO: 28.

47. The genetically-modified yeast of any one of claims 1-46, comprising at least one additional copy of one or more of the exogenous polynucleotides encoding the cytokinin hydroxylase, NADPH-cytochrome P450 reductase and cytokinin riboside 5′-monophosphate phosphoribohydrolase.

48. The genetically-modified yeast of claim 47, comprising an additional copy of each of the exogenous polynucleotides encoding the cytokinin hydroxylase, NADPH-cytochrome P450 reductase and cytokinin riboside 5′-monophosphate phosphoribohydrolase.

49. The genetically-modified yeast of claim 48, wherein the additional copies of the exogenous polynucleotides encoding the cytokinin hydroxylase, NADPH-cytochrome P450 reductase and cytokinin riboside 5′-monophosphate phosphoribohydrolase are arranged in a single polynucleotide molecule.

50. The genetically-modified yeast of claim 49, wherein the single polynucleotide molecule comprising the nucleic acid sequence set forth in SEQ ID NO: 26.

51. The genetically-modified yeast of claim 27, comprising: an exogenous polynucleotide comprising the sequence set forth in SEQ ID NO: 25 and an exogenous polynucleotide comprising the sequence set forth in SEQ ID NO: 28.

52. The genetically-modified yeast of claim 51, further comprising an exogenous polynucleotide comprising the sequence set forth in SEQ ID NO: 26.

53. A method for producing at least one cytokinin selected from the group consisting of trans-zeatin, trans-zeatin riboside, isopentenyladenine isopentenyladenine riboside and any combination thereof, the method comprising:
(A) providing a yeast genetically-modified according to any one of claims 1-52;
(B) culturing the genetically-modified yeast in a culture medium under conditions suitable for expression of the exogenous polynucleotides; and
(C) recovering the at least one cytokinin from the medium.

54. The method for producing at least one cytokinin of claim 53, wherein step (A) comprises providing a genetically-modified yeast in which the exogenous polynucleotides are expressed under a galactose-inducible promoter, and wherein the culturing in step (B) is performed by fed-batch fermentation process comprising a glucose growth phase followed by a galactose induction phase with intermittent ethanol pulses.

55. The method for producing at least one cytokinin of any one of claims 53-54, wherein said method results in the production of a plurality of cytokinins comprising at least 60% (w/w) trans-zeatin and isopentenyladenine out of the total amount of the plurality of cytokinins.

56. The method for producing at least one cytokinin of claim 55, wherein at least 70% (w/w) of the plurality of cytokinins are trans-zeatin and isopentenyladenine.

57. An expression cassette for genetically-modifying a yeast, comprising at least one regulatory element operably-linked to a nucleic acid sequence selected from the group consisting of:
(a) a nucleic acid sequence encoding an isopentenyl transferase;
(b) a nucleic acid sequence encoding a cytokinin hydroxylase;
(c) a nucleic acid sequence encoding an NADPH-cytochrome P450 reductase; and
(d) a nucleic acid sequence encoding a cytokinin riboside 5′-monophosphate phosphoribohydrolase.

58. The expression cassette of claim 57, wherein the nucleic acid sequence encodes an enzyme selected from the group consisting of plant enzymes, fungal enzymes, homologs thereof and any combination thereof.

59. The expression cassette of claim 58, wherein the nucleic acid sequence encodes a plant enzyme or a homolog thereof.

60. The expression cassette of claim 59, wherein the plant is Arabidopsis thaliana.

61. The expression cassette of claim 60, wherein the nucleic acid sequence is selected from the group consisting of:
(a) a nucleic acid sequence encoding A. thaliana IPT4 comprising the sequence set forth in SEQ ID NO: 8;
(b) a nucleic acid sequence encoding A. thaliana CYP735A1 comprising the sequence set forth in SEQ ID NO: 9;
(c) a nucleic acid sequence encoding A. thaliana ATR1 comprising the sequence set forth in SEQ ID NO: 10; and
(d) a nucleic acid sequence encoding A. thaliana LOG7 comprising the sequence set forth in SEQ ID NO: 11.

62. The expression cassette of claim 57 or claim 61, wherein the at least one regulatory element comprises an inducible promoter.

63. The expression cassette of 62, wherein the promoter is a galactose-inducible promoter.

64. The expression cassette of 63, selected from the group consisting of:
an expression cassette for expressing A. thaliana IPT4 comprising the sequence set forth in SEQ ID NO: 15;
an expression cassette for expressing A. thaliana CYP735A1 comprising the sequence set forth in SEQ ID NO: 16;
an expression cassette for expressing A. thaliana ATR1 comprising the sequence set forth in SEQ ID NO: 17; and
an expression cassette for expressing A. thaliana LOG7 comprising the sequence set forth in SEQ ID NO: 18.

65. A nucleic acid construct for genetically-modifying a yeast to produce cytokinins comprising a plurality of expression cassettes, the plurality of expression cassettes comprising: an expression cassette for expressing A. thaliana IPT4 comprising the sequence set forth in SEQ ID NO: 15; an expression cassette for expressing A. thalianaCYP735A1 comprising the sequence set forth in SEQ ID NO: 16; an expression cassette for expressing A. thaliana ATR1 comprising the sequence set forth in SEQ ID NO: 17; and an expression cassette for expressing A. thaliana LOG7 comprising the sequence set forth in SEQ ID NO: 18.

66. A vector for transforming a yeast cell to produce cytokinins, the vector comprising the nucleic acid construct of claim 65, flanked by 5′ and 3′ yeast genomic integrating sequences.

67. The vector of claim 66, comprising a sequence selected from SEQ ID NO: 29 and SEQ ID NO: 30.

68. A method for generating a yeast that produces at least one cytokinin selected from the group consisting of trans-zeatin, trans-zeatin riboside, isopentenyladenine isopentenyladenine riboside and any combination thereof, the method comprising transforming the yeast with a plurality of exogenous polynucleotides according to any one of claims 57-64.

69. The method of claim 68, further comprising transforming the yeast with at least one additional exogenous polynucleotide selected from the group consisting of: (A) one or more exogenous polynucleotide encoding an amino-terminal truncated HMG-COA reductase lacking the transmembrane domain; and (B) an exogenous polynucleotide encoding an isopentenyl isomerase.

70. The method of claim 69, wherein the yeast is transformed with:
an exogenous polynucleotide encoding an amino-terminal truncated HMG-COA reductase 1 having at least 80% identity to the amino acid sequence of Sc-tHMG1;
an exogenous polynucleotide encoding an amino-terminal truncated HMG-COA reductase 1 having at least 80% identity to the amino acid sequence of Xanthophyllomyces dendrorhous amino-terminal truncated HMG-COA reductase 1 (Xd-tHMG1); and
an exogenous polynucleotide encoding isopentenyl isomerase having at least 80% identity to the amino acid sequence of S. cerevisiae isopentenyl isomerase IDI1.

71. The method of claim 70, wherein the yeast is transformed with: an exogenous polynucleotide encoding Sc-tHMG1 comprising the nucleic acid sequence set forth in SEQ ID NO: 12; an exogenous polynucleotide encoding Xd-tHMG1 comprising the nucleic acid sequence set forth in SEQ ID NO: 13; and an exogenous polynucleotide encoding S. cerevisiae IDI1 comprising the nucleic acid sequence set forth in SEQ ID NO: 14.

72. The method of any one of claims 69-71, further comprising transforming the yeast with an additional copy of each of the exogenous polynucleotides encoding the cytokinin hydroxylase, NADPH-cytochrome P450 reductase and cytokinin riboside 5′-monophosphate phosphoribohydrolase.

73. The method of any one of claims 68-72, wherein the yeast is Saccharomyces cerevisiae.
